(19) United States
(12) Patent Application Publication
(10) Pub. No.: US 2025/0345523 A1
(43) Pub. Date: Nov. 13, 2025

(54) System and Method for Fluid Delivery Using Multiple Fluid Delivery Devices

(71) Applicant: Becton, Dickinson and Company, Franklin Lakes, NJ (US)

(72) Inventors: Martin M. Coyne, III, Towaco, NJ (US); Andrew Mark Bryant, Buggingen (DE); Matthew Conner D'Auria, Bernardsville, NJ (US); Amy Catherine Rinaldi, Morristown, NJ (US); Katherine Ford Brigham, Rutherford, NJ (US); Peter Skutnik, Midland Park, NJ (US); Garrett Austin Sheffer, Hoboken, NJ (US)

(21) Appl. No.: 18/867,846

(22) PCT Filed: Aug. 4, 2023
(86) PCT No.: PCT/US2023/029502

§ 371 Date: Nov. 21, 2024

Related U.S. Application Data
(60) Provisional application No. 63/395,511, filed on Aug. 5, 2022.

(51) Int. Cl. A61M 5/31; A61M 5/00

(52) U.S. Cl. CPC A61M 5/31; A61M 5/002; A61M 2005/3125; A61M 2205/33; A61M 2205/3576; A61M 2205/502

(57) ABSTRACT

A system for fluid delivery using multiple fluid delivery devices may include a plurality of modules associated with a plurality of fluid delivery devices. Each module of the plurality of modules may include: at least one processor, a wireless communication device configured to establish a wireless communication network with at least one other wireless communication device of at least one other module of the plurality of modules, at least one sensor configured to sense a state of a fluid delivery device associated with that module, and/or a user feedback device configured to provide feedback associated with one or more states of one or more fluid delivery devices of the plurality of fluid delivery devices.

### CROSS REFERENCE TO RELATED APPLICATION

This application claims priority to U.S. Provisional Patent Application No. 63/395,511, filed Aug. 5, 2022, the disclosure of which is hereby incorporated by reference in its entirety.

### BACKGROUND

Existing fluid delivery devices, such as autoinjectors (e.g., the BD Physioject™, etc.), on-body injectors (OBIs) (e.g., the BD Libertas™, etc.), and/or the like may provide start-of-dose (SOD) feedback, in-process feedback (IPF), and/or end-of-dose (EOD) feedback. SOD, IPF, and EOD feedback may include audible, tactile, and/or visual indicators to accommodate a wide user population with one or more physical and/or cognitive limitations. Notably, these existing fluid delivery devices display the status of a unitary device, as is expected with discrete injections with a single device.

Serial use of multiple autoinjectors may be indicated for all patients or a subset thereof, depending on indication or response. For example, AJOVY® (fremanezumab-vfrm) provided by Teva Pharmaceuticals USA, Inc. instructs use of one autoinjector once monthly (Q1M) or three autoinjectors (1.5 mL fill volume each; 675 mg total dose) once quarterly (Q3M). Cosentyx® (secukinumab) provided by Novartis Pharmaceuticals Corporation instructs use of two 150 mg (1 mL) autoinjector devices once monthly (Q1M) for plaque psoriasis and one 150 mg (1 mL) autoinjector device for ankylosing spondylitis once monthly (Q1M). Thus, with the indicated use of multiple autoinjector devices, because the device actuation is discrete and the injection time is short, the device instructions for use (IFU) typically suffice to avoid errors in administration. This is demonstrated by regulatory approval of multiple instances of multiple autoinjector devices in various configurations per dosing interval.

An example of the use of multiple OBI devices includes ULTROMIRIS® (ravulizumab-cwvz) provided by Alexion Pharmaceuticals, Inc., which announced results of a Phase 3 trial of SC ravulizumab using two 3.5 mL West SmartDose® devices per dosing interval. The 3.5 mL West SmartDose® device is commercially available, and has already been incorporated into an approved presentation of Repatha® (evolocumab) provided by Amgen Inc. in a single-device (420 mg/3.5 mL) presentation. In this large volumes of medications are being delivered, but at dosing intervals more similar to autoinjector devices.

### SUMMARY

Accordingly, provided are improved systems, devices, products, apparatus, and/or methods for fluid delivery using multiple fluid delivery devices.

According to some non-limiting embodiments or aspects, provided is a system, including: a plurality of modules associated with a plurality of fluid delivery devices, wherein each module of the plurality of modules includes: at least one processor, a wireless communication device configured to establish a wireless communication network with at least one other wireless communication device of at least one other module of the plurality of modules, at least one sensor configured to sense a state of a fluid delivery device associated with that module, and a user feedback device configured to provide feedback associated with one or more states of one or more fluid delivery devices of the plurality of fluid delivery devices.

In some non-limiting embodiments or aspects, for each module, in response to being activated, that module is configured to: determine whether that module is a first module of the plurality of modules to be activated; in response to determining that that module is the first module to be activated, establish that module as a master module; and in response to determining that that module is not the first module to be activated, establish, with that module as a slave module, the wireless communication network with the master module.

In some non-limiting embodiments or aspects, for each module, that module is configured to be activated in response to an initiation of a fluid delivery procedure with the fluid delivery device associated with that module.

In some non-limiting embodiments or aspects, in response to activating the plurality of modules associated with the plurality of fluid delivery devices, the plurality of modules associated with the plurality of fluid delivery devices is configured to include a master module associated with a first fluid delivery device and at least one slave module associated with at least one second fluid delivery device, wherein the master module is configured to delay providing, via the user feedback devices of the master module and the at least one slave module, (i) feedback indicating that a first fluid delivery procedure associated with the first fluid delivery device is complete and (ii) feedback indicating that at least one second fluid delivery procedure associated with the at least one second fluid delivery device is complete until (i) a state of the first fluid delivery device sensed by the master module includes a completion of the first fluid delivery procedure and (ii) a state of the at least one second fluid delivery device sensed by the at least one slave module includes a completion of the at least one second fluid delivery.

In some non-limiting embodiments or aspects, in response to activating the plurality of modules associated with the plurality of fluid delivery devices, the master module is configured to control the user feedback devices of the master module and the at least one slave module to provide a same feedback indicating that a fluid injection is in progress until (i) the state of the first fluid delivery device sensed by the master module includes the completion of the first fluid delivery procedure and (ii) the state of the at least one second fluid delivery device sensed by the at least one slave module includes a completion of the at least one second fluid delivery.

In some non-limiting embodiments or aspects, the plurality of modules associated with the plurality of fluid delivery devices includes a first module associated with a first fluid delivery device and a second module associated with a second fluid delivery device, wherein, in response to an activation of the first module, the first module is configured to automatically provide, via the user feedback device of the first module, feedback indicating that the first fluid delivery device is ready for use in a first fluid delivery procedure, and wherein, in response to an activation of the second module, the second module is configured to automatically establish the wireless communication network with the first module.

In some non-limiting embodiments or aspects, in response to sensing that the state of the first fluid delivery device includes a completion of the first fluid delivery procedure, the first module is configured to communicate to the second module a message indicating that the first fluid delivery procedure is complete, wherein, in response to receiving the message indicating that the first fluid delivery procedure is complete, the second module is configured to automatically provide, via the user feedback device of the second module, feedback indicating that the second fluid delivery device is ready for use in a second fluid delivery procedure.

In some non-limiting embodiments or aspects, the second module is configured to provide no feedback via the user feedback device of the second module until the second module receives the message indicating that the first fluid delivery procedure is complete.

In some non-limiting embodiments or aspects, in response to sensing that the state of the first fluid delivery device includes an initiation of the first fluid delivery procedure, the first module is configured to automatically provide, via the user feedback device of the first module, feedback indicating that the first fluid delivery procedure is initiated and/or a progression of the first fluid delivery procedure.

In some non-limiting embodiments or aspects, the plurality of modules associated with the plurality of fluid delivery devices further includes at least one third module associated with at least one third fluid delivery device, wherein, in response to sensing that the state of the second fluid delivery device includes a completion of the second fluid delivery procedure, the second module is configured to communicate to the at least one third module a message indicating that the second fluid delivery procedure is complete, wherein, in response to receiving the message indicating that the second fluid delivery procedure is complete, the at least one third module is configured to automatically provide, via the user feedback device of the at least one third module, feedback indicating that the at least one third fluid delivery device is ready for use in at least one third fluid delivery procedure.

In some non-limiting embodiments or aspects, the system further includes: a package housing the first module, the first fluid delivery device, the second module, and the second fluid delivery device, wherein each of the first module and the second module is configured to be automatically activated in response to an opening of the package and/or a removal of that module from the package.

In some non-limiting embodiments or aspects, the plurality of fluid delivery devices includes a plurality of on-body injectors.

In some non-limiting embodiments or aspects, each module of the plurality of modules further includes a housing that houses the at least one processor, the wireless communication device, the at least one sensor, and the user feedback device, and wherein, for each module, the housing of that module is attached to a further housing of the fluid delivery device associated with that module.

In some non-limiting embodiments or aspects, for each module, the housing of that module covers a further user feedback device of the fluid delivery device associated with that module such that the further user feedback device is inhibited from providing feedback to a user.

In some non-limiting embodiments or aspects, each module of the plurality of modules is integrated with the fluid delivery device associated with that module such that the at least one processor, the wireless communication device, the at least one sensor, and the user feedback device are included in a same housing of the fluid delivery device associated with that module.

In some non-limiting embodiments or aspects, the wireless communication network includes an ad-hoc mesh network.

In some non-limiting embodiments or aspects, each module of the plurality of modules is configured to sense as the state of the fluid delivery device associated with that module at least one of the following: an initiation of a fluid delivery procedure, a progression of the fluid delivery procedure, a completion of the fluid delivery procedure, or any combination thereof.

In some non-limiting embodiments or aspects, the plurality of modules is configured to coordinate, via the wireless communication network, the feedback provided via the user feedback device of each module based on the sensed state of each fluid delivery device of the plurality of fluid delivery devices.

In some non-limiting embodiments or aspects, the plurality of modules is configured to delay providing via the user feedback device of each module an indication that the fluid delivery procedure associated with that module is complete until the fluid delivery procedure associated with each module of the plurality of modules is complete.

In some non-limiting embodiments or aspects, the plurality of modules associated with the plurality of fluid delivery devices includes a first module associated with a first fluid delivery device and a second module associated with a second fluid delivery device, and wherein the second module is configured to provide no feedback via the user feedback device of the second module until the second module receives, from the first module via the wireless communication network, a message indicating that the fluid delivery procedure associated with the first module is complete.

In some non-limiting embodiments or aspects, the feedback includes a playback of a recorded verbal message associated with the one or more states of the one or more fluid delivery devices of the plurality of fluid delivery devices.

In some non-limiting embodiments or aspects, one or more modules of the plurality of modules is configured to apply one or more feedback rules to the one or more states of the one or more fluid delivery devices of the plurality of fluid delivery devices to determine at least one module of the plurality of modules for providing the feedback associated with the one or more states of the one or more fluid delivery devices of the plurality of fluid delivery devices.

In some non-limiting embodiments or aspects, the one or more feedback rules include at least one of the following feedback rules: a first feedback rule that a module of the plurality of modules associated with a fluid delivery device with a slower or longer delivery provides an end of dose feedback, a second feedback rule that a module of the plurality of modules associated with a first activated fluid delivery device of plurality of fluid delivery devices provides a start of dose feedback, or any combination thereof.

In some non-limiting embodiments or aspects, the at least one sensor includes at least one of the following sensors: a start of dose sensor configured to sense a start of a dose by the fluid delivery device associated with that module, a dose progression sensor configured to sense a progression of the dose by the fluid delivery device associated with that module, an end of dose sensor configured to sense an end of the dose by the fluid delivery device associated with that module, a dose time and/or duration sensor configured to sense a time and/or a duration of the dose by the fluid delivery device associated with that module, a temperature sensor configured to sense a temperature of a fluid to be delivered by the fluid delivery device associated with that module, a needle shield removal sensor configured to sense a removal of a needle shield of the fluid delivery device associated with that module, an accelerometer configured to sense an orientation of the module and/or the fluid delivery device associated with that module, or any combination thereof.

In some non-limiting embodiments or aspects the at least one processor is configured to provide, as input to a machine learning model, the one or more states of the one or more fluid delivery devices of the plurality of fluid delivery devices, and receive, as output from the machine learning model, the feedback associated with the one or more states of the one or more fluid delivery devices of the plurality of fluid delivery devices.

In some non-limiting embodiments or aspects, the playback of the recorded verbal message provides at least one of the following: an individual dose status of one or more fluid delivery devices of the plurality of fluid delivery devices; a combined dose status of the plurality of fluid delivery devices; a next user step; an instruction and/or a cautionary statement; a reminder for a next dose; or any combination thereof.

According to some non-limiting embodiments or aspects, provided is a system for a single device dosing regimen with verbal feedback, including: a module associated with a fluid delivery device, wherein the module includes: at least one processor, at least one sensor configured to sense a state of the fluid delivery device, and a user feedback device configured to playback a recorded verbal message associated with the state of the fluid delivery device.

In some non-limiting embodiments or aspects, the at least one sensor includes at least one of the following sensors: a start of dose sensor configured to sense a start of a dose by the fluid delivery device, a dose progression sensor configured to sense a progression of the dose by the fluid delivery device, an end of dose sensor configured to sense an end of the dose by the fluid delivery device, a dose time and/or duration sensor configured to sense a time and/or a duration of the dose by the fluid delivery device, a temperature sensor configured to sense a temperature of a fluid to be delivered by the fluid delivery device, a needle shield removal sensor configured to sense a removal of a needle shield of the fluid delivery device, an accelerometer configured to sense an orientation of the module and/or the fluid delivery device, or any combination thereof.

In some non-limiting embodiments or aspects, the at least one processor is configured to provide, as input to a machine learning model, the state of the fluid delivery device, and receive, as output from the machine learning model, the recorded verbal message associated with the state of the fluid delivery device.

In some non-limiting embodiments or aspects, the playback of the recorded verbal message provides at least one of the following: a dose status of the fluid delivery device; a next user step; an instruction and/or a cautionary statement; a reminder for a next dose; or any combination thereof.

According to some non-limiting embodiments or aspects, provided is a method, including: establishing, with a plurality of modules associated with a plurality of fluid delivery devices, a wireless communication network including the plurality of modules; for each module of the plurality of modules, sensing, with at least one sensor of that module, a state of a fluid delivery device of the plurality of fluid delivery devices associated with that module; and providing, with one or more modules of the plurality of modules, feedback associated with one or more states of one or more fluid delivery devices of the plurality of fluid delivery devices.

In some non-limiting embodiments or aspects, the method further includes: for each module, in response to being activated: determining, with that module, whether that module is a first module of the plurality of modules to be activated; in response to determining that that module is the first module to be activated, establishing, with that module, that module as a master module; and in response to determining that that module is not the first module to be activated, establishing, with that module as a slave module, the wireless communication network with the master module.

In some non-limiting embodiments or aspects, for each module, that module is activated in response to an initiation of a fluid delivery procedure with the fluid delivery device associated with that module.

In some non-limiting embodiments or aspects, the method further includes: activating the plurality of modules associated with the plurality of fluid delivery devices, wherein, in response to activating the plurality of modules associated with the plurality of fluid delivery devices, the plurality of modules associated with the plurality of fluid delivery devices includes a master module associated with a first fluid delivery device and at least one slave module associated with at least one second fluid delivery device; and delaying, with the master module, providing, via the master module and the at least one slave module, (i) feedback indicating that a first fluid delivery procedure associated with the first fluid delivery device is complete and (ii) feedback indicating that at least one second fluid delivery procedure associated with the at least one second fluid delivery device is complete until (i) a state of the first fluid delivery device sensed by the master module includes a completion of the first fluid delivery procedure and (ii) a state of the at least one second fluid delivery device sensed by the at least one slave module includes a completion of the at least one second fluid delivery.

In some non-limiting embodiments or aspects, the method further includes: in response to activating the plurality of modules associated with the plurality of fluid delivery devices, controlling, with the master module, the master module and the at least one slave module to provide a same feedback indicating that a fluid injection is in progress until (i) the state of the first fluid delivery device sensed by the master module includes the completion of the first fluid delivery procedure and (ii) the state of the at least one second fluid delivery device sensed by the at least one slave module includes a completion of the at least one second fluid delivery.

In some non-limiting embodiments or aspects, the plurality of modules associated with the plurality of fluid delivery devices includes a first module associated with a first fluid delivery device and a second module associated with a second fluid delivery device, and wherein the method further includes: activating the first module; in response to activating of the first module, automatically providing, with the first module, feedback indicating that the first fluid delivery device is ready for use in a first fluid delivery procedure; activating the second module; and in response to activating the second module, automatically establishing, with the second module as a slave module, the wireless communication network with the first module as a master module.

In some non-limiting embodiments or aspects, the method further includes: sensing, with the first module, the state of the first fluid delivery device includes a completion of the first fluid delivery procedure; in response to sensing that the state of the first fluid delivery device includes a completion of the first fluid delivery procedure, communicating, with the first module, to the second module via the wireless communication network, a message indicating that the first fluid delivery procedure is complete; and in response to receiving the message indicating that the first fluid delivery procedure is complete, automatically providing, with the second module, feedback indicating that the second fluid delivery device is ready for use in a second fluid delivery procedure.

In some non-limiting embodiments or aspects, the second module provides no feedback until the second module receives the message indicating that the first fluid delivery procedure is complete.

In some non-limiting embodiments or aspects, the method further includes: sensing, with the first module, that the state of the first fluid delivery device includes an initiation of the first fluid delivery procedure; in response to sensing that the state of the first fluid delivery device includes an initiation of the first fluid delivery procedure, automatically providing, with the first module, feedback indicating that the first fluid delivery procedure is initiated and/or a progression of the first fluid delivery procedure.

In some non-limiting embodiments or aspects, the plurality of modules associated with the plurality of fluid delivery devices further includes at least one third module associated with at least one third fluid delivery device, and wherein the method further includes: sensing, with the second module, that the state of the second fluid delivery device includes a completion of the second fluid delivery procedure; in response to sensing that the state of the second fluid delivery device includes a completion of the second fluid delivery procedure, communicating, with the second module, to the at least one third module via the wireless communication network, a message indicating that the second fluid delivery procedure is complete; and in response to receiving the message indicating that the second fluid delivery procedure is complete, automatically providing, with the at least one third module, feedback indicating that the at least one third fluid delivery device is ready for use in at least one third fluid delivery procedure.

In some non-limiting embodiments or aspects, a package houses the first module, the first fluid delivery device, the second module, and the second fluid delivery device, and wherein each of the first module and the second module is automatically activated in response to an opening of the package and/or a removal of that module from the package.

In some non-limiting embodiments or aspects, the plurality of fluid delivery devices includes a plurality of on-body injectors.

In some non-limiting embodiments or aspects, each module of the plurality of modules includes a housing that houses at least one processor, a wireless communication device, at least one sensor, and a user feedback device, and wherein, for each module, the housing of that module is attached to a further housing of the fluid delivery device associated with that module.

In some non-limiting embodiments or aspects, for each module, the housing of that module covers a further user feedback device of the fluid delivery device associated with that module such that the further user feedback device is inhibited from providing feedback to a user.

In some non-limiting embodiments or aspects, each module of the plurality of modules is implemented by the fluid delivery device associated with that module.

In some non-limiting embodiments or aspects, the wireless communication network includes an ad-hoc mesh network.

In some non-limiting embodiments or aspects, the method further includes: for each module of the plurality of modules, sensing, with that module, as the state of the fluid delivery device associated with that module at least one of the following: an initiation of a fluid delivery procedure, a progression of the fluid delivery procedure a completion of the fluid delivery procedure, or any combination thereof.

In some non-limiting embodiments or aspects, the method further includes: coordinating, with the plurality of modules, via the wireless communication network, the feedback provided via each module based on the sensed state of each fluid delivery device of the plurality of fluid delivery devices.

In some non-limiting embodiments or aspects, the method further includes: delaying, with the plurality of modules, providing via each module an indication that the fluid delivery procedure associated with that module is complete until the fluid delivery procedure associated with each module of the plurality of modules is complete.

In some non-limiting embodiments or aspects, the plurality of modules associated with the plurality of fluid delivery devices includes a first module associated with a first fluid delivery device and a second module associated with a second fluid delivery device, and wherein the second module provides no feedback until the second module receives, from the first module via the wireless communication network, a message indicating that the fluid delivery procedure associated with the first module is complete.

In some non-limiting embodiments or aspects, providing, with the one or more modules of the plurality of modules, the feedback associated with the one or more states of the one or more fluid delivery devices of the plurality of fluid delivery devices includes playing back a recorded verbal message associated with the one or more states of the one or more fluid delivery devices of the plurality of fluid delivery devices.

In some non-limiting embodiments or aspects, the method further includes: applying, with one or more modules of the plurality of modules, one or more feedback rules to the one or more states of the one or more fluid delivery devices of the plurality of fluid delivery devices to determine at least one module of the plurality of modules for providing the feedback associated with the one or more states of the one or more fluid delivery devices of the plurality of fluid delivery devices.

In some non-limiting embodiments or aspects, the one or more feedback rules include at least one of the following feedback rules: a first feedback rule that a module of the plurality of modules associated with a fluid delivery device with a slower or longer delivery provides an end of dose feedback, a second feedback rule that a module of the plurality of modules associated with a first activated fluid delivery device of plurality of fluid delivery devices provides a start of dose feedback, or any combination thereof.

In some non-limiting embodiments or aspects, the at least one sensor includes at least one of the following sensors: a start of dose sensor configured to sense a start of a dose by the fluid delivery device associated with that module, a dose progression sensor configured to sense a progression of the dose by the fluid delivery device associated with that module, an end of dose sensor configured to sense an end of the dose by the fluid delivery device associated with that module, a dose time and/or duration sensor configured to sense a time and/or a duration of the dose by the fluid delivery device associated with that module, a temperature sensor configured to sense a temperature of a fluid to be delivered by the fluid delivery device associated with that module, a needle shield removal sensor configured to sense a removal of a needle shield of the fluid delivery device associated with that module, an accelerometer configured to sense an orientation of the module and/or the fluid delivery device associated with that module, or any combination thereof.

In some non-limiting embodiments or aspects, providing, with the one or more modules of the plurality of modules, the feedback associated with the one or more states of the one or more fluid delivery devices of the plurality of fluid delivery devices includes providing, as input to a machine learning model, the one or more states of the one or more fluid delivery devices of the plurality of fluid delivery devices, and receiving, as output from the machine learning model, the feedback associated with the one or more states of the one or more fluid delivery devices of the plurality of fluid delivery devices.

In some non-limiting embodiments or aspects, playing back the recorded verbal message associated with the one or more states of the one or more fluid delivery devices of the plurality of fluid delivery devices provides at least one of the following: an individual dose status of the one or more fluid delivery devices of the plurality of fluid delivery devices; a combined dose status of the plurality of fluid delivery devices; a next user step; an instruction and/or a cautionary statement; a reminder for a next dose; or any combination thereof.

According to some non-limiting embodiments or aspects, provided is a method for a single device dosing regimen with verbal feedback, comprising: sensing, with at least one sensor of a module, a state of a fluid delivery device associated with that module; and playing back, with a speaker of the module, a recorded verbal message associated with the state of the fluid delivery device.

In some non-limiting embodiments or aspects, the at least one sensor includes at least one of the following sensors: a start of dose sensor configured to sense a start of a dose by the fluid delivery device, a dose progression sensor configured to sense a progression of the dose by the fluid delivery device, an end of dose sensor configured to sense an end of the dose by the fluid delivery device, a dose time and/or duration sensor configured to sense a time and/or a duration of the dose by the fluid delivery device, a temperature sensor configured to sense a temperature of a fluid to be delivered by the fluid delivery device, a needle shield removal sensor configured to sense a removal of a needle shield of the fluid delivery device, an accelerometer configured to sense an orientation of the module and/or the fluid delivery device, or any combination thereof.

In some non-limiting embodiments or aspects, the method further includes: providing, with the at least one processor, as input to a machine learning model, the one or more states of the one or more fluid delivery devices of the plurality of fluid delivery devices; and receiving, with the at least one processor, as output from the machine learning model, the recorded verbal message associated with the state of the fluid delivery device.

In some non-limiting embodiments or aspects, the playback of the recorded verbal message provides at least one of the following: a dose status of the fluid delivery device; a next user step; an instruction and/or a cautionary statement; a reminder for a next dose; or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of non-limiting embodiments or aspects of an environment in which systems, devices, products, apparatus, and/or methods, described herein, can be implemented;

FIG. 2 is a diagram of non-limiting embodiments or aspects of components of one or more devices and/or one or more systems of FIG. 1;

FIG. 3A is a perspective view of an implementation of non-limiting embodiments or aspects of the present disclosure;

FIG. 3B is an exploded view of the implementation shown in FIG. 3A showing a module separated from a fluid delivery device;

FIG. 3C is a front perspective view of the implementation shown in FIGS. 3A and 3B showing various states of example feedback;

FIG. 4A illustrates non-limiting embodiments or aspects of a serial fluid delivery method and a parallel fluid delivery method using multiple fluid delivery devices;

FIG. 4B illustrates non-limiting embodiments or aspects of example feedback;

FIG. 5 illustrates an exemplary process, in accordance with non-limiting embodiments or aspects of the present disclosure.

FIG. 6 is a signal flow diagram of an implementation of non-limiting embodiments or aspects of a process for fluid delivery using multiple fluid delivery devices in a parallel configuration;

FIG. 7 is a signal flow diagram of an implementation non-limiting embodiments or aspects of a process for fluid delivery using multiple fluid delivery devices in a serial configuration;

FIG. 8 is a perspective and side views of non-limiting embodiments or aspects of a module and fluid delivery device assembly;

FIG. 9 is a top view of non-limiting embodiments or aspects of a module and fluid delivery device assembly;

FIG. 10 is side views of non-limiting embodiments or aspects of a module and fluid delivery device assembly;

FIG. 11 is a side and top view of non-limiting embodiments or aspects of a module and fluid delivery device assembly;

FIG. 12 is perspective view of non-limiting embodiments or aspects of a package for multiple module and fluid delivery device assemblies;

FIG. 13 is a perspective view of non-limiting embodiments or aspects of a package for multiple module and fluid delivery device assemblies;

FIG. 14 is a perspective view of non-limiting embodiments or aspects of a package for multiple module and fluid delivery device assemblies;

FIG. 15 is a perspective view of non-limiting embodiments or aspects of a package for multiple module and fluid delivery device assemblies;

FIG. 16 is a perspective view of non-limiting embodiments or aspects of a package for multiple module and fluid delivery device assemblies.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

A need for multiple fluid delivery devices, such as multiple OBI devices, and/or the like may be dictated by device readiness (e.g., technical maturity, etc.), pharmacokinetic drivers of a specific molecule (which drive delivered volume requirements per dosing interval), and/or changes in volume during clinical development (e.g., unexpected formulation or bioavailability challenges, also affecting delivered volume requirements, etc.).

Multiple fluid delivery devices, such as multiple OBI devices, and/or the like may be used for a delivery volume that is greater than a volume that a single standard fluid delivery device can deliver. For example, the BD Libertas™ is available in 5 mL and 10 mL variants, but not in a variant larger than 10 mL. If a dose larger than 10 mL is to be provided in a single dosing interval, and the BD Libertas™ is a preferred device, multiple devices may be used. As an example, a dose of 10 mL (2×5 mL) or a dose of 20 ml (2×10 mL) may use a serial delivery method (e.g. fluid delivery with device “A” followed by fluid delivery with device “B”) or a parallel delivery method (e.g., fluid delivery with device “A” concurrent with fluid delivery with device “B”), which may enable a pharmaceutical customer who has performed device evaluation and/or container stability work to remain on the preferred BD Libertas™ device platform and/or avoid evaluation of alternative devices (e.g., the Enable enFuse® 25 mL or 50 mL variants, etc.).

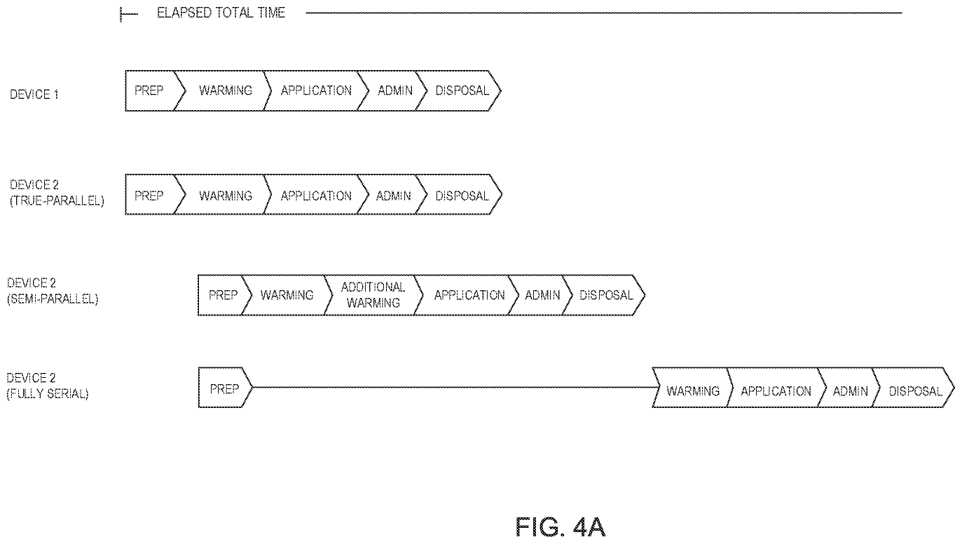

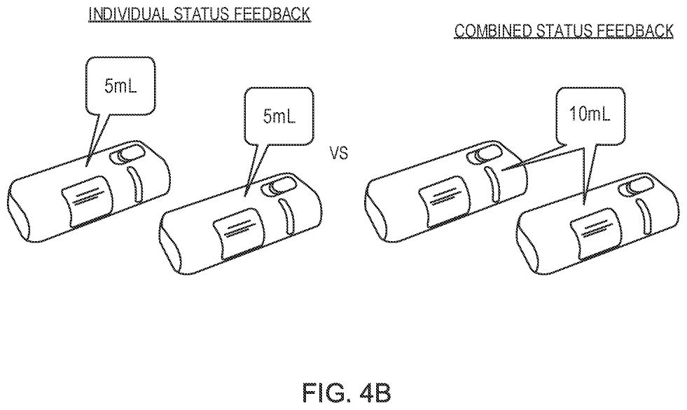

Referring now to FIG. 4A, FIG. 4A illustrates non-limiting embodiments or aspects of a serial fluid delivery method and a parallel fluid delivery method using multiple fluid delivery devices. “Serial” or “parallel” may refer to the sequence of application and activation of a plurality of fluid delivery devices. Non-limiting embodiments or aspects of the present disclosure may include serial and/or parallel application or use of multiple fluid delivery devices, such as multiple OBI devices, and/or the like. As is discussed below, a parallel or a serial application option may be selected for use based on specific clinical administration scenarios and/or molecule needs.

When multiple OBI devices are used, there may be a risk of user confusion with parallel application of the multiple OBI devices (e.g., each device delivering a dose at the same time, etc.) in which the user mistakes one ‘end-of-dose signal’ for the other. For example, this may occur in semi-parallel administration, when the devices are activated at slightly different times (e.g., with a delay or gap in between the device activations), and/or this confusion may lead to use errors. As an example, a situation to avoid is when a user, hearing or feeling an end-of-dose feedback from a first OBI device, confuses which OBI device is finished and removes a second OBI device before the second OBI device has completed delivering the full dose, which may result in an underdose to a patient (e.g., a so-called “wet” injection, etc.).

Serial administration may reduce the risk of use errors, as each device may be independently used, and more closely resembles examples of using multiple existing autoinjectors per dosing interval. However, as the injection times are significantly longer than those for autoinjector devices (e.g., minutes-to-hours versus seconds, etc.), there may also be use errors. For example, a user may not understand the serial intention of the workflow and may forget to use subsequent devices and/or use the devices at different times (e.g., a day or a week later, etc.). Thus, improved solutions are needed to facilitate proper serial administration with multiple OBI devices.

It is noted that although non-limiting embodiments or aspects of the present disclosure may be described primly with respect to two OBI devices, (e.g., two BD Libertas™ devices, etc.), non-limiting embodiments or aspects of the present disclosure are not limited to two devices, to OBI devices, or to the BD Libertas™, and non-limiting embodiments or aspects of the present disclosure may include and/or use three or more devices and/or fluid delivery devices other than OBI devices and/or the BD Libertas™. For example, the BD Libertas™ may be primarily intended for at-home administration by an untrained user, such as a patient themselves, a family caregiver, and/or the like. However, non-limiting embodiments or aspects of the present disclosure are not specific to a specific OBI design, at home administration, and/or an untrained user and/or may be used by patients, caregivers, and/or health care provides, whether at home or in clinic, as a same unmet need may exist in each of these contexts.

OBIs may offer increased volume capacity whether used independently, or as a plurality of devices per dosing interval, which may enable administration of a wider variety of molecules (e.g., molecules that cannot be up-concentrated further, etc.) and/or may extend duration between doses. Such a use case may be different from that of existing devices described herein, such as Repatha® (evolocumab) provided by Amgen Inc. and ULTROMIRIS® (ravulizumab-cwvz) provided by Alexion Pharmaceuticals, Inc., which have dosing intervals similar to autoinjector devices. In contrast, longer dosing intervals reduce an opportunity for spaced repetition of device use steps, thereby increasing a risk that users do not remember how to use the device properly. For example, for longer dosing intervals (e.g., Q2M, Q3M, Q6M, etc.), each dose time may be akin to a first time using the device, particularly for less capable users. This situation may be compounded when multiple devices are used, and better solutions are needed to enable users to successfully perform more complex steps potentially less frequently. For example, there is a need for coordinating feedback across multiple devices to ensure usability of the system, even when the OBI devices may be highly usable in isolation.

Non-limiting embodiments or aspects of the present disclosure are directed to apparatus, systems, and/or methods for fluid delivery using multiple fluid delivery devices that include a plurality of modules associated with a plurality of fluid delivery devices. Each module of the plurality of modules may include: a processor, a wireless communication device configured to establish a wireless communication network with at least one other wireless communication device of at least one other module of the plurality of modules, a sensor configured to sense a state of a fluid delivery device associated with that module, and/or a user feedback device configured to provide feedback associated with one or more states of one or more fluid delivery devices of the plurality of fluid delivery devices. For example, non-limiting embodiments or aspects of the present disclosure relate to using multiple fluid delivery devices, such as on body injectors (OBIs) (e.g., the BD Libertas™, etc.), and/or the like to deliver larger doses of fluid or medication at a specific injection interval. As an example, non-limiting embodiments or aspects of the present disclosure may coordinate start-of-dose (SOD) feedback, in-process feedback (IPF), and/or end-of-dose (EOD) feedback between fluid delivery devices and/or provide additional layers of feedback to a user of the system to improve usability, as well as provide affordances to encourage a specific administration scheme (e.g., serial, parallel, etc.) using packaging, labeling, and/or on-device indicators, thereby allowing simple pharmaceutical company customization of a platform fluid delivery device while reducing, minimizing, or eliminating changes to the fluid delivery device.

In this way, non-limiting embodiments or aspects of the present disclosure may provide improved apparatus, systems, and/or methods that may mitigate potential usability issues associated with a user employing multiple OBI devices during a single dosing interval in a variety of clinically relevant administration scenarios and/or settings. Further, non-limiting embodiments or aspects of the present disclosure may provide apparatus, systems, and/or methods that may be selectively included with minimal (or no) change to an OBI device, allowing relatively complex administration scenarios. Further, non-limiting embodiments or aspects of the present disclosure may address an increased complexity of using multiple OBIs and/or different use models compared to simpler, existing multiple-autoinjector use cases. Although OBIs may be generally highly usable in isolation, the introduction of multiple OBIs may make attaching, observing, interpreting, and/or removing devices more complex. For example, additional cognitive load may be placed on the user, and the potential for use errors may increase through cognitive overload.

Accordingly, an advantage of non-limiting embodiments or aspects of the present disclosure, whether used for serial or parallel administration, may include establishing device order in an ad hoc manner via mesh networking of modules/fluid delivery devices as individual modules/devices are activated, which may accommodate a wide variety of use cases, providing flexibility in order of devices activated and/or timing of device activation. In manufacturing, an advantage of non-limiting embodiments or aspects of the present disclosure may include accommodation of all use cases with a same module architecture through drug-specific modifications to firmware of modules/fluid deliver devices, thereby easing customization while retaining efficiency of manufacture and/or component sourcing.

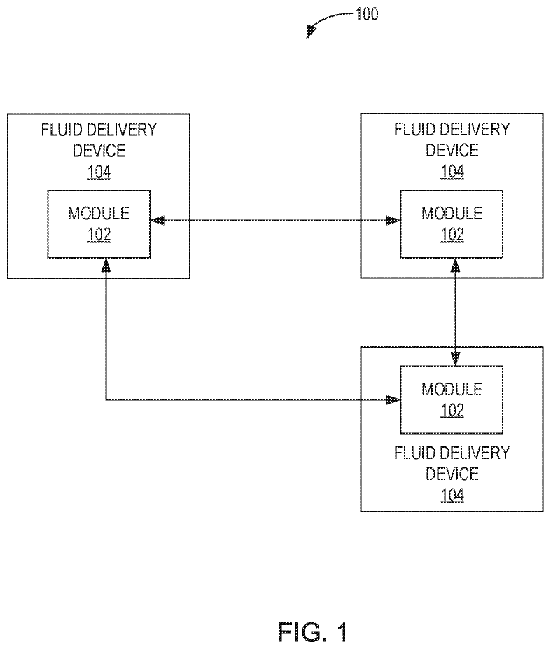

Referring now to FIG. 1, FIG. 1 is a diagram of an example environment 100 in which devices, systems, methods, and/or products described herein, may be implemented. As shown in FIG. 1, environment 100 includes a plurality of modules 102 associated with (e.g., associated with, corresponding to, connected to, integrated with, etc.) a plurality of fluid delivery devices 104. For example, a single module 102 may be connected to and/or integrated with each fluid delivery device 104 to form a module and fluid delivery device assembly 102, 104. In some non-limiting embodiments or aspects, a single standalone module 102 may be associated with (e.g., associated with, corresponding to, connected to, integrated with, etc.) a single standalone fluid delivery device 104 (e.g., for a single device dosing regimen, etc.).

A module 102 may include one or more devices capable of receiving information and/or data from one or more other modules of the plurality of modules 102 via a wireless communication connection (e.g., an NFC communication connection, a Bluetooth® communication connection, a Zigbee communication connection, etc.) and/or communicating information and/or data to the one or delivery more other modules of the plurality of modules 102 via a wireless communication connection (e.g., an NFC communication connection, a Bluetooth® communication connection, a Zigbee communication connection, etc.). For example, the plurality of modules 102 may be configured to form a wireless communication network. As an example, the plurality of modules 102 may be configured to form an ad-hoc mesh network (e.g., a partially connected mesh network, a fully connected mesh network, etc.) in which individual modules 102 form nodes of the mesh network.

A fluid delivery device 104 may include an autoinjector (e.g., the BD Physioject™, etc.), an on-body injector (OBI) (e.g., the BD Libertas™, etc.), and/or the like. For example, a fluid delivery device 104 may include a housing configured for receiving a container at least partially filled with a medical fluid, and a drive mechanism (e.g., a stopper, a plunger, etc.) associated with the housing configured for delivering the medical fluid from the container to a patient in a dosing or fluid delivery procedure. As an example, a fluid delivery device 104 may include a wearable injection and/or infusion device as disclosed by U.S. patent application Ser. No. 15/942,804, filed on Apr. 2, 2018, and/or a drug delivery system 10 as disclosed by U.S. Pat. No. 10,549,044, issued on Feb. 4, 2020, the disclosures of which are incorporated herein by reference in their entireties.

The number and arrangement of systems and devices shown in FIG. 1 is provided as an example. There can be additional systems and/or devices, fewer systems and/or devices, different systems and/or devices, or differently arranged systems and/or devices than those shown in FIG. 1. Furthermore, two or more systems or devices shown in FIG. 1 can be implemented within a single system or a single device, or a single system or a single device shown in FIG. 1 can be implemented as multiple, distributed systems or devices. Additionally, or alternatively, a set of systems or a set of devices (e.g., one or more systems, one or more devices, etc.) of environment 100 can perform one or more functions described as being performed by another set of systems or another set of devices of environment 100.

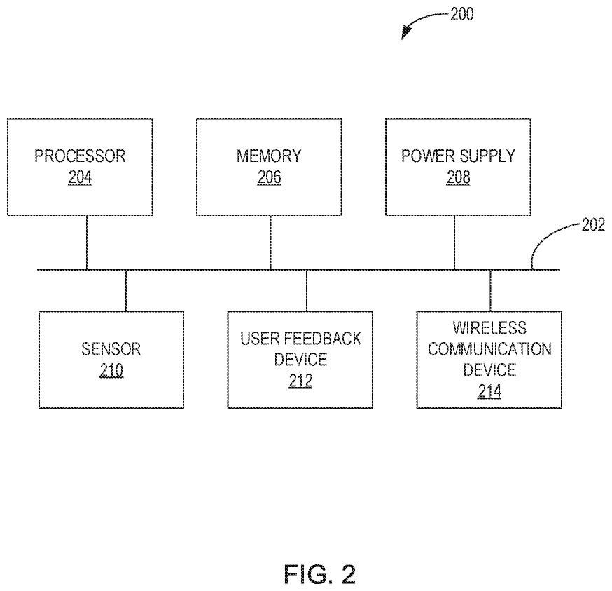

Referring now to FIG. 2, FIG. 2 is a diagram of example components of a device 200. Device 200 may correspond to a module 102 (e.g., one or more devices of a system of a module 102, etc.), to a fluid delivery device 104 (e.g., one or more devices of a system of a fluid delivery device 104, etc.), and/or to a module and fluid delivery device assembly 102, 104. In some non-limiting embodiments or aspects, a module 102 (e.g., one or more devices of a system of a module 102, etc.) and/or a fluid delivery device 104 (e.g., one or more devices of a system of a fluid delivery device 104, etc.) may include at least one device 200 and/or at least one component of device 200. As shown in FIG. 2, device 200 may include bus 202, processor 204, memory 206, power supply 108, sensor 210, user feedback device 212, and/or wireless communication device 214. In some non-limiting embodiments or aspects, one or more components of device 200 may be arranged on a printed circuit board (PCB).

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), a low power microcontroller unit (MCU), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Power supply 208 may be configured to power bus 202, processor 204, memory 206, sensor 210, user feedback device 212, and/or wireless communication device 214. For example, power supply 208 may include a battery (e.g., a rechargeable battery, a disposable battery, etc.), an energy harvester (e.g., an energy harvester configured to derive energy from one or more external sources, such as electromagnetic energy, solar energy, thermal energy, wind energy, salinity gradients, kinetic energy, and/or the like, etc.), or any combination thereof.

Sensor 210 of module 102 associated with fluid delivery 104 may include at least one of the following sensors: a start of dose sensor configured to sense a start of a dose by the fluid delivery device 104 associated with that module 102, a dose progression sensor configured to sense a progression of the dose by the fluid delivery device 104 associated with that module 102, an end of dose sensor configured to sense an end of the dose by the fluid delivery device 104 associated with that module 102, a dose time (and/or date) and/or duration sensor configured to sense a time and/or a duration of the dose by the fluid delivery device 104 associated with that module 102, a temperature sensor configured to sense a temperature of a fluid to be delivered by the fluid delivery device 104 associated with that module 102 (e.g., a temperature sensor configured to sense whether a temperature of the medication is ready or comfortable for injection, etc.), a needle shield removal sensor configured to sense a removal of a needle shield of the fluid delivery device 104 associated with that module 102, an accelerometer configured to sense an orientation of the module 102 and/or the fluid delivery device 104 associated with that module 102, or any combination thereof. For example, sensor 210 may include one or more of the sensors and/or detection switches disclosed by U.S. patent application Ser. No. 15/942,804, filed on Apr. 2, 2018, the disclosure of which is incorporated herein by reference in its entirety. As an example, sensor 210 may include one or more sensors such as an environmental sensor(s) (e.g. a temperature sensor, etc.), to both improve the dose detection algorithms (e.g. fluid viscosity temperature effects) and/or to provide feedback to the user (e.g. drug is too cold for injection, etc.); a sensor(s) to detect mechanical motions associated with injector operation (e.g. switches to detect activation, completion, needle insertion/withdrawal, and/or other device events and states); one or more sensors to continuously monitor dose delivery, such as an optical sensor array, a capacitive sensor array, an inductive sensor array, and/or the like; detection switches including mechanical components (e.g., with direct mechanical interactions with underlying components of fluid delivery device 104, etc.), and/or infrared-based optical sensors (e.g. reflectance or photointerrupter sensors, etc.) to allow non-contact detection, to detect mechanical motions associated with changes in fluid delivery device state, such as needle shield removal, injector unlock, activation button depression, injection activation, dose progression injection completion, and/or the like; and/or the like.

In some non-limiting embodiments or aspects, because opening a package may activate a module, the module may use a temperature sensor to sense the temperature of each device once the package is opened, and the module may compare a temperature of the cartridge or container including the fluid or medication to an ambient temperature to determine if the medication is comfortable to inject (e.g., room temperature, etc.).

User feedback device 212 may include a component that provides output information from device 200 (e.g., a display, a speaker, one or more light-emitting diodes (LEDs), a haptic device, etc.). In some non-limiting embodiments or aspects, user feedback device 212 may include a component that permits device 200 to receive information, such as via user input (e.g., a touch screen display, a keyboard, a keypad, a mouse, a button, a switch, a microphone, etc.). Additionally or alternatively, user feedback device 212 may include a sensor for sensing information (e.g., a global positioning system (GPS) component, an accelerometer, a gyroscope, an actuator, etc.).

Wireless communication device 214 may include a transceiver-like component (e.g., a transceiver, a separate receiver and transmitter, etc.) that enables device 200 to communicate with other devices via a wireless connection. Wireless communication device 214 may permit device 200 to receive information from another device and/or provide information to another device. For example, wireless communication device 214 may include one or more computing devices, chips, contactless transmitters, contactless transceivers, NFC transmitters, RFID transmitters, Bluetooth Transceivers®, Zigbee transceivers, and/or the like that enable wireless communication device 214 to receive information directly from and/or communicate information directly to another device (e.g., another wireless communication device 214, another module 102, an external computing device, etc.) via a short range wireless communication connection (e.g., a communication connection that uses NFC protocol, a communication connection that uses Radio-frequency identification (RFID), a communication connection that uses a Bluetooth® wireless technology standard, a communication connection that uses a Zigbee wireless technology standard, etc.).

Device 200 may perform one or more processes described herein. Device 200 may perform these processes based on processor 204 executing software instructions stored by a computer-readable medium, such as memory 206. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 from another computer-readable medium or from another device via wireless communication device 214. When executed, software instructions stored in memory 206 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 may include data storage or one or more data structures (e.g., a database, etc.). Device 200 may be capable of receiving information from, storing information in, communicating information to, or searching information stored in the data storage or one or more data structures in memory 206.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

In some non-limiting embodiments or aspects, each module of the plurality of modules 102 (and/or a single standalone module 102 for a single device dosing regimen) includes: a processor 204, a wireless communication device 214 configured to establish a wireless communication network with at least one other wireless communication device of at least one other module of the plurality of modules 102, a sensor 210 configured to sense a state of a fluid delivery device associated with that module, and/or a user feedback device 212 configured to provide feedback associated with one or more states of one or more fluid delivery devices of the plurality of fluid delivery devices 104. In such an example, the plurality of fluid delivery devices 102 may include a plurality of on-body injectors, and/or the wireless communication network may include an ad-hoc mesh network formed by the plurality of modules 102.

In some non-limiting embodiments or aspects, user feedback device 212 may be configured to provide feedback associated with and/or based on one or more states of one or more fluid delivery devices of the plurality of fluid delivery devices 104 by playing back (e.g., via a speaker, etc.) a recorded verbal message (e.g., by playing back recorded audio that provides an audible verbal prompt or instruction to the user when played back, etc.). For example, module 102 may store (e.g., in memory 206, etc.) a plurality of recorded verbal messages. As an example, sensor 210 may monitor a state of a fluid delivery device 104 associated with that module (e.g., a temperature, a dose status, an error status, etc.) and/or wireless communication device 214 may receive one or more other states of one or more other fluid delivery devices 104 from one or more other modules 102, and processor 204 may control user feedback device 212 to playback a recorded verbal message associated with and/or based on the state of the fluid delivery device 104 associated with that module and/or the one or more states of the one or more other fluid delivery devices 104 received from the one or more other modules 102. In such an example, processor 204 may use a look-up table and/or one or more rules to determine the recorded verbal message associated with the state of the fluid delivery device 104 associated with that module and/or the one or more states of the one or more other fluid delivery devices 104 received from the one or more other modules 102.

A recorded verbal message may include at least one of the following: a recorded verbal message providing a dose status of the fluid delivery device 104 associated with that module, a dose status of the one or more other fluid delivery devices 104, and/or a combined dose status of the fluid delivery device 104 and the one or more other fluid delivery devices 104 (e.g., a recorded verbal message indicating a device ready/not ready for dosing status, a recorded message indicating a dose is in progress, a recorded verbal message indicating an amount of a dose progression, a recorded verbal message indicating a dose is complete, a recorded verbal message indicating a dosing error has occurred, etc.); a recorded verbal message providing a next user step(s) (e.g., a recorded verbal message to remove a needle shield, a recorded verbal message instructing the user to choose a particular injection site on the user's body, a recorded verbal message to apply the device to the selected injection site, a recorded verbal instruction to remove and/or dispose of the device, etc.; an instruction(s), and/or a cautionary statement(s) (e.g., a recorded verbal message to inspect the device for damage, wash the user's hands, and/or check and care for the injection site on the user's body post injection, a recorded verbal message to wait a predetermined period of time before dosing); a recorded verbal message providing other information, such as a reminder for a next dose, information associated with insurance and/or reimbursements, any combination thereof; and/or the like. In such an example, a recorded verbal message may apply to a single standalone fluid delivery device, to an individual fluid delivery device of the plurality of fluid delivery devices, and/or to multiple fluid delivery devices of the plurality of fluid delivery devices. For example, playback of a recorded verbal message may provide at least one of the following: an individual dose status of one or more fluid delivery devices of the plurality of fluid delivery devices; a combined dose status of the plurality of fluid delivery devices; a next user step; an instruction and/or a cautionary statement; a reminder for a next dose; information associated with insurance and/or reimbursements, or any combination thereof.

Referring again to FIG. 4A and also referring to FIG. 4B, a plurality of serial administration and/or parallel administration module and fluid delivery device assemblies 102, 104 may each individually provide a recorded verbal message indicting a dose delivery status (e.g., 4 mL of 5 mL has been delivered by the individual device, etc.) of that respective module and fluid delivery device assembly 102, 104 and/or a single module and fluid delivery device assembly 102, 104 (or each module and fluid delivery device assembly 102, 104) may provide a recorded verbal message indicating a combined dose delivery status for each of the plurality of serial or parallel module and fluid delivery device assemblies 102, 104 (e.g., 8 mL of 10 mL has been delivered by the combined devices, etc.). In such an example, because slight variations may exist in an injection time for a delivered dose of a same amount of fluid between the plurality of serial and/or parallel module and fluid delivery device assemblies 102, 104, one or more modules 102 (or each module) of the plurality of modules 102 may store (e.g., in memory 206, etc.) one or more feedback rules and/or apply the one or more feedback rules to one or more states of one or more fluid delivery devices of the plurality of fluid delivery devices to determine at least one module of the plurality of modules for providing the feedback associated with the one or more states of the one or more fluid delivery devices of the plurality of fluid delivery devices (e.g., to determine which one or more of the plurality of serial and/or parallel module and fluid delivery device assemblies 102, 104 feedback is provided from, etc.). For example, the one or more feedback rules may include at least one of the following rules: a rule that a module and fluid delivery device assembly 102, 104 with a slower or longer delivery provides an end of dose feedback (e.g., a recorded verbal message indicating an end of dose, etc.), a rule that a first activated module and fluid delivery device assembly 102, 104 provides a start of dose feedback (e.g., a recorded verbal message indicating a start of dose, etc.), and/or the like. In such an example, an injection time may be an amount of time elapsed between the first start of dose and the last end of dose. If different dosing regimens are prescribed to be delivered in multiple serial doses, recorded verbal messages may be provided by one or more modules 102 to indicate when a next dose is due and/or which fluid delivery device of the plurality of fluid delivery devices 104 should be activated to provide the next dose.

In some non-limiting embodiments or aspects, a recorded verbal message may correspond to one or more non-verbal feedbacks (e.g., a visual indicator, haptic feedback, etc.). For example, a recorded verbal message may be provided in place of or in addition to one or more corresponding non-verbal feedbacks. In some non-limiting embodiments or aspects, a recorded verbal message may provide alternative and/or additional information as compared to one or more non-verbal feedbacks (e.g., a visual indicator feedback, a haptic feedback, etc.), such as information at more frequent intervals, information including verbal cues prompted by specific user/device interaction(s) and/or device orientation(s), and/or information synching and consolidating verbal cues across multiple module and fluid delivery device assemblies 102, 104 operating in a serial administration configuration and/or a parallel administration or configuration. As an example, a recorded verbal message may be provided in response to module 102 powering on, in response sensor 210 (e.g., a needle shield removal sensor, etc.) sensing a removal of a needle shield of a fluid delivery device 104, in response to sensor 210 (e.g., an accelerometer, etc.) sensing a predetermined orientation of a module and fluid delivery device assembly 102, 104 (e.g., in response to sensing that the assembly 102, 104 has been flipped over and therefore that the user is probably about to remove the needle shield, etc.), and/or the like

In some non-limiting embodiments or aspects, processor 204 of module 102 may be configured to provide, as input to a machine learning model, the one or more states of the one or more fluid delivery devices of the plurality of fluid delivery devices, and receive, as output from the machine learning model, the feedback associated with the one or more states of the one or more fluid delivery devices of the plurality of fluid delivery devices. For example, processor 204 of module 102 may use machine learning to continually improve interpretation of user interactions with the plurality of fluid delivery devices 104 to determine how, when, and/or which feedback (e.g., which recorded verbal message, etc.) is provided and/or from which one or more modules the feedback is provided. As an example, module 102 may store the machine learning model in memory 206 and/or remotely access the machine learning model via wireless communication device 214.

In such an example, module 102 may determine the feedback associated with the one or more states of the one or more fluid delivery devices of the plurality of fluid delivery devices based on a machine learning technique (e.g., a pattern recognition technique, a data mining technique, a heuristic technique, a supervised learning technique, an unsupervised learning technique, etc.). For example, module 102 and/or a remote computing device may generate a model (e.g., an estimator, a classifier, a prediction model, etc.) based on a machine learning algorithm (e.g., a decision tree algorithm, a gradient boosted decision tree algorithm, a neural network algorithm, a convolutional neural network algorithm, etc.). In such an example, module 102 and/or the remote computing device may determine the feedback associated with the one or more states of the one or more fluid delivery devices of the plurality of fluid delivery devices.

Module 102 and/or the remote computing device may generate the model based on training data including one or more states of one or more fluid delivery devices. For example, the model may be designed to receive, as an input, one or more states of one or more fluid delivery devices, and provide, as an output, the feedback associated with the one or more states of the one or more fluid delivery devices. As an example, module 102 and/or the remote computing device may analyze the training data using machine learning techniques to generate the model (e.g., a prediction model, a classification model, a recommendation model, etc.). The machine learning techniques may include, for example, supervised and/or unsupervised techniques, such as decision trees (e.g., gradient boosted decision trees), logistic regressions, artificial neural networks (e.g., convolutional neural networks), Bayesian statistics, learning automata, Hidden Markov Modeling, linear classifiers, quadratic classifiers, association rule learning, and/or the like. In some non-limiting embodiments or aspects, module 102 and/or the remote computing device generates or trains the model using the machine learning techniques to optimize an objective or loss function (e.g., an objective or loss function that depends on an output of the model and/or one or more labels for the input, etc.). In some non-limiting embodiments or aspects, module 102 and/or the remote computing device may stores the trained model (e.g., stores the trained model for later use). In some non-limiting embodiments or aspects, module 102 and/or the remote computing device stores the trained model in a data structure (e.g., a database, a linked list, a tree, etc.). In some non-limiting embodiments or aspects, the data structure is located within module 102 and/or the remote computing device may or external (e.g., remote from) module 102 and/or the remote computing device may.

In some non-limiting embodiments or aspects, module 102 may be configured to receive (e.g., via wireless communication device, etc.), record (e.g., via a microphone of user feedback device 212, etc.), and/or store (e.g., in memory 206) one or more custom recorded verbal messages and/or one or more custom feedback rules. For example, a provider, such as a medical professional prescribing and/or issuing module and fluid delivery device assembly 102, 104 may upload (e.g., via a supporting application on a computing device, etc.) and/or record (e.g., via the microphone, etc.) and/or store (e.g., in memory 206, etc.) one or more custom recorded verbal messages and/or custom feedback rules (e.g., after manufacturing, at a time of issue and/or prescription, etc.).

It is noted that although recorded verbal messages according to non-limiting embodiments or aspects of the present disclosure may be described primarily with respect to multiple devices, (e.g., multiple devices in a serial administration configuration or a parallel administration configuration, etc.), recorded verbal messages according to non-limiting embodiments or aspects of the present disclosure are not limited to use with multiple devices, and non-limiting embodiments or aspects of the present disclosure may include a single standalone device that uses recorded verbal messages (e.g., that uses recorded verbal messages for a single device dosing regimen, etc.).

For example, a single standalone module 102 may associated with a single standalone fluid delivery device 104 for a single device dosing regimen. Components and/or functions of a single standalone module 102 may be the same as or similar to components of a module 102 configured for use in in a serial administration configuration or a parallel administration configuration. Components and/or functions of a standalone fluid delivery device may be the same as or similar to components of a fluid delivery device configured for use in in a serial administration configuration or a parallel administration configuration. Components and/or functions of a single standalone module and fluid delivery device assembly 102, 104 may be the same as or similar to components of a module and fluid delivery device assembly 102, 104 configured for use in in a serial administration configuration or a parallel administration configuration. In some non-limiting embodiments or aspects, a single, standalone module may include a module such as described by U.S. patent application Ser. No. 15/942,804, filed on Apr. 2, 2018, the disclosure of which is incorporated herein by reference in its entirety.

As an example, non-limiting embodiments or aspects of a system for a single device dosing regimen with verbal feedback may include a module 102 associated with a fluid delivery device 104. The module 102 may include: at least one processor (e.g., processor 204, etc.), at least one sensor (e.g., sensor 206, etc.) configured to sense a state of the fluid delivery device 104, and a user feedback device 212 configured to provide feedback associated with the state of the fluid delivery device 104. The feedback may include a playback of a recorded verbal message associated with the state of the fluid delivery device. In such an example, module 102 may determine the feedback For example, user feedback device 212 may be configured to provide feedback associated with and/or based on one or more states of the fluid delivery device 104 by playing back (e.g., via a speaker, etc.) a recorded verbal message (e.g., by playing back recorded audio that provides an audible verbal prompt or instruction to the user when played back, etc.). As an example, module 102 may store (e.g., in memory 206, etc.) a plurality of recorded verbal messages. In such an example, sensor 210 may monitor a state of the fluid delivery device 104 (e.g., a temperature, a dose status, an error status, etc.), and processor 204 may control user feedback device 212 to playback a recorded verbal message associated with and/or based on the state of the fluid delivery device 104 (e.g., by accessing a look-up table and/or applying one or more rules to the state of the fluid delivery device. For example, playback of a recorded verbal message may provide at least one of the following: a dose status of the fluid delivery device; a next user step; an instruction and/or a cautionary statement; a reminder for a next dose; information associated with insurance and/or reimbursements, or any combination thereof.

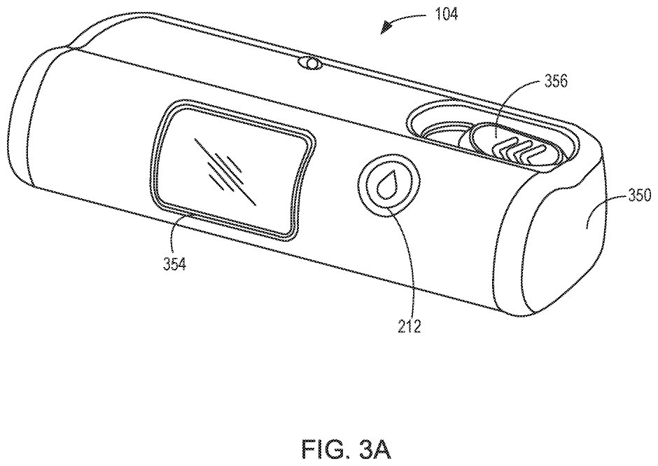

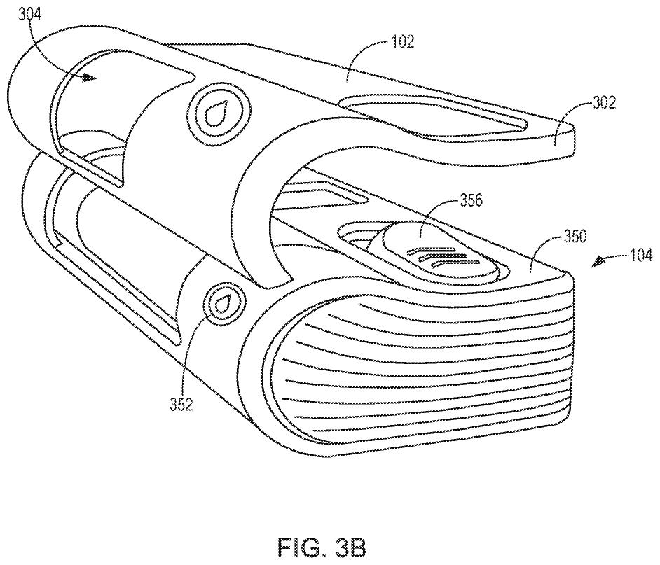

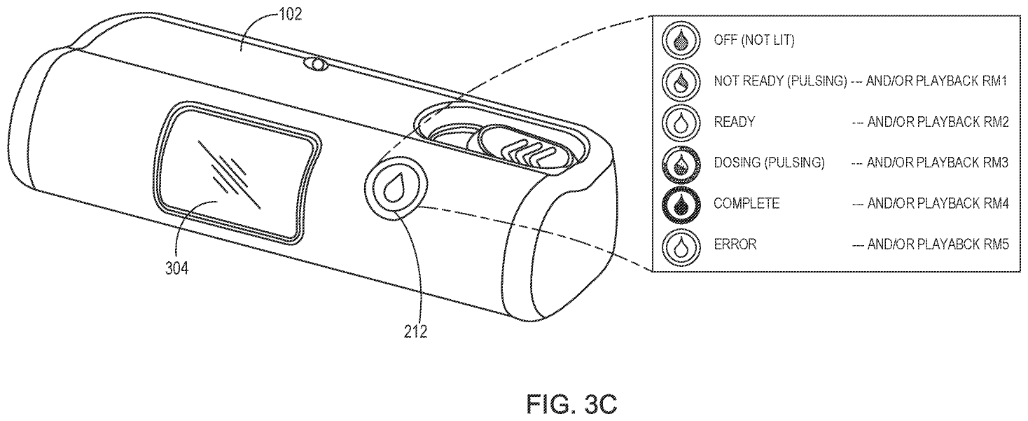

In some non-limiting embodiments or aspects, a module 102 includes a housing that houses components of the module (e.g., one or more components of device 200, etc.), and the housing of the module is attached to a housing of the fluid delivery device 104 associated with that module, for example, to form a module and fluid delivery device assembly 102, 104. For example, and referring also to FIGS. 3A and 3B, a module (e.g., each module, etc.) of the plurality of modules 102 may further include a housing 302 that houses the processor 204, the wireless communication device 214, the sensor 210, and/or the user feedback device 212, and/or, for the module (e.g., each module, etc.), the housing 302 of that module may be attached (e.g., removably attached, permanently attached, etc.) to a further housing 350 of the fluid delivery device 104 associated with that module (e.g., housing 102 of wearable injection and/or infusion device 100 disclosed in U.S. patent application Ser. No. 15/942,804, etc.), for example, to form a module and fluid delivery device assembly 102, 104. As an example, a module 102 may be provided as a separately manufactured snap-on component positioned and retained over a top outer surface (e.g., away from or opposite the patient's skin, etc.) of a fluid delivery device 104. For example, a module 102 may be provided for each fluid delivery device 104 and/or may be pre-assembled to each fluid delivery device 104 during secondary or tertiary assembly, and/or the module 102 and fluid delivery device 104 assembly may be packaged as described herein to further improve intuitiveness and/or communicate that each assembly 102, 104 provided in the packaging is for use in a single dosing interval. Positioning of sensor(s) 210 within each module 102 may be preconfigured to monitor one or more aspects of operation of a specific fluid delivery device 104 to which that module is to be attached and/or integrated.

In some non-limiting embodiments or aspects, and still referring to FIGS. 3A and 3B, for a module (e.g., for each module, etc.), a housing 302 of a module 102 may cover (e.g., partially cover, completely cover, etc.) a further user feedback device 352 of a fluid delivery device 104 associated with that module 102 such that the further user feedback device 352 is inhibited (e.g., partially inhibited, completely inhibited, etc.) from providing feedback to a user when the module 102 is attached to and/or integrated with the fluid delivery device 104. For example, the housing 302 of the module 102 may hide, replace, and/or cover existing mechanically-driven feedback of the fluid delivery device 104 associated with that module 102 and/or hide, replace, and/or cover existing electronically-provided feedback of the fluid delivery device 104 associated with that module 102. The housing 302 of the module 102 may include a viewing window 304 for viewing contents of fluid delivery device 104 (e.g., contents of a medication container, etc.) through an existing viewing window 354 of the fluid delivery device 104 and/or provide access to an injector activation button 356 configured to activate the fluid delivery device 104. Additionally, or alternatively, firmware or indicator circuitry in electronically-provided devices may be modified to inhibit the device from providing feedback to a user, for example, when a module 102 is completely integrated with and/or implemented by a fluid delivery device 104. In this way, clear and simple feedback may be provided to a user (e.g., conflicting signals to the user may be avoided, etc.).

Referring now to FIG. 3C, FIG. 3C is a front perspective view of the implementation shown in FIGS. 3A and 3B showing various states of example feedback. As shown in FIG. 3C, user feedback device 212 may operate a visual indicator in a first manner (e.g., to visually indicate the device is not ready, etc.) and/or play back a first recorded verbal message RM1 after module 102 is activated to instruct the user to remove module and fluid delivery device assembly 102, 104 from a package, to check an expiry date, to inspect the assembly for damage, to safely discard the assembly and select a new assembly if the assembly is damaged, and/or to wait a predetermined time (e.g., thirty minutes, etc.) before activating the fluid delivery device. In such an example, user feedback device 212 may periodically play back additional first recorded messages RM1 indicating an amount of the predetermined time remaining before the user may activate the device. In response to expiry of the predetermined time, user feedback device 212 may operate the visual indicator in a second manner (e.g., to indicate that the device is ready for dosing, etc.) different than the first manner and/or play back a second recorded verbal message RM2 to instruct the user to choose an injection site on the abdomen or thigh, to slowly pull the needle cover and peel of the adhesive liner, to adhere the assembly to the chosen location and run a finger along the edge of the pad, to activate the dose by first sliding and then pushing the activation button when ready, and to indicate that the user will hear an audible “click” and the status of the visual indicator will change in response to pushing the activation button. During delivery of the dose, user feedback device 212 may operate the visual indicator in a third manner (e.g., to visually indicate dose progression, etc.) different than the first manner and the second manner and/or play back a third recorded verbal message RM3 to indicate to the user that once the dose is completed, the visual indicator will change and a second audible click will be heard and to remove and discard the device and call the user's healthcare professional if the user notices dose progression has not changed in 20 minutes. In such an example, user feedback device 212 may periodically play back additional third recorded messages RM3 indicating an amount of time remaining before the dose is complete. In response to completion of the dose, user feedback device 212 may operate the visual indicator in a fourth manner (e.g., to visually indicate dose completion, etc.) different than the first manner, the second manner, and the third manner and/or play back a fourth recorded verbal message RM4 to indicate to the user that the dose has been successfully delivered and that the user may remove and safely dispose of the device. In response to an error occurring during dosing, user feedback device 212 may operate the visual indicator in a fifth manner (e.g., to visually indicate an error has occurred, etc.) and/or play back a fifth recorded verbal message RM5 to indicate to the user that an error has occurred. In such an example, a specific fifth recorded verbal message RM5 of a plurality of fifth recorded verbal messages associated with a type of the error and/or instructions to self-correct or to remove and discard the device and contact a health care profession may be played back by user feedback device 212.

Accordingly, non-limiting embodiments or aspects of the present disclosure may replace existing mechanically and/or electrically-driven visual user feedback in a fluid delivery device 104 with a module 102 that senses a state of the fluid delivery device 104 (e.g., by detecting a movement of a plunger in the fluid delivery device 104, etc.) and/or provides feedback, such as an electronic indicator (e.g., a LED, etc.) audible output (e.g., a recorded message, etc.), corresponding to different states of one or more of the fluid delivery devices 104 that form the wireless communication network.

In some non-limiting embodiments or aspects, a module 102 is integrated with and/or implemented by a fluid delivery device 104 associated with that module such that components of the module 102 (e.g., one or more components of device 200, etc.) are included in a same housing of the fluid delivery device 104 associated with that module (e.g., housing 102 of wearable injection and/or infusion device 100 disclosed in U.S. patent application Ser. No. 15/942,804, etc.) and/or one or more components of the fluid delivery device 104 associated with that module may perform one or more functions described as being performed by that module, for example, to form a module and fluid delivery device assembly 102, 104. For example, each module of the plurality of modules 102 may be integrated with and/or implemented by the fluid delivery device 104 associated with that module such that the processor 204, the wireless communication device 214, the sensor 210, and/or the user feedback device 212 are included in a same housing of the fluid delivery device 104 associated with that module. As an example, each module of the plurality of modules 102 may be implemented by the fluid delivery device 104 associated with that module 102.

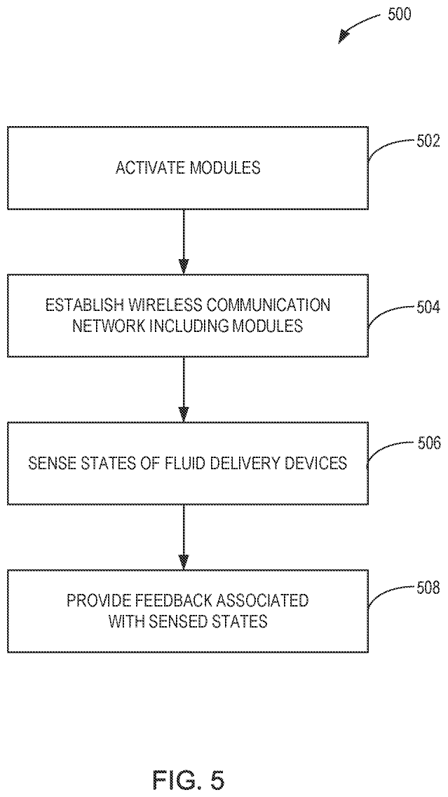

FIG. 5 illustrates an exemplary process, in accordance with non-limiting embodiments or aspects of the present disclosure.

As shown in FIG. 5, at step 502, process 500 includes activating a plurality of modules. For example, a user may activate a plurality of modules 102 associated with a plurality of fluid delivery devices 104 (e.g., a plurality of assemblies 102, 104, etc.). As an example, each module of the plurality of modules 102 may be activated (e.g., turned on, powered up, woke up from a sleep state, caused to initiate processing with processor 204 of program instructions associated with one or more processes described herein, etc.) by a user in any sequence. In such an example, as described herein with respect to FIG. 2, each module of the plurality of modules 102 may include: a processor 204, a wireless communication device 214 configured to establish a wireless communication network with at least one other wireless communication device of at least one other module of the plurality of modules 102, a sensor 210 configured to sense a state of a fluid delivery device associated with that module, and a user feedback device 212 configured to provide feedback associated with one or more states of one or more fluid delivery devices of the plurality of fluid delivery devices 104. It is noted that an actuation of a fluid delivery device 104 and/or an assembly 102, 104 to initiate delivery of a fluid and/or an injection procedure may be distinguished from an activation of a module 102 and/or an assembly 102, 104 in which the module 102 and/or the assembly 102, 104 may powered on, begin processing, and/or the like.

In some non-limiting embodiments or aspects, the plurality of modules 102 associated with the plurality of fluid delivery devices 104 (e.g., the plurality of assemblies 102, 104, etc.) may be preconfigured (e.g., during manufacturing, by a healthcare provider issuing and/or prescribing the assemblies 102, 104 via a supporting application on a computing device, with software instructions, etc.) to operate according to a serial and/or a parallel configuration in which a type of user feedback (e.g., visual, haptic, recorded verbal messages, etc.), a source of the user feedback (e.g., a particular module(s) of the plurality of modules 102 providing the user feedback, etc.), a timing of the user feedback (e.g., a predetermined period, a predetermined frequency, a preconfigured user feedback provided in response to one or more predetermined states of one or more fluid delivery devices of the plurality of fluid delivery devices, etc.), a content of the user feedback (e.g., a prompt or instruction, a warning, a custom recorded verbal message, etc.), a master/slave configuration between the modules 102, or any combination thereof is preconfigured according to a desired configuration and/or determined according to a preconfigured order of the plurality of assemblies 102, 104, a position of a particular assembly 102, 104 in the preconfigured order of the plurality of assemblies 102, 104, a number of the plurality of assemblies 102, 104, a type of one or more assemblies 102, 104 of the plurality of assemblies 102, 104 (e.g., a dose volume, an injection rate, a type of medication, etc.), or any combination thereof. In this way, the plurality of assemblies 102, 104 and/or the user feedback provided therefrom may be tailored to a particular dosing regimen in order to reduce user confusion and/or errors.

In some non-limiting embodiments or aspects, a package may house the plurality of modules 102 and the plurality of fluid delivery devices 104 (e.g., a first module, a first fluid delivery device, a second module, a second fluid delivery device, etc.). Each of the modules of the plurality of modules 102 (e.g., the first module, the second module, etc.) may be configured to be automatically activated in response to an opening of the package and/or a removal of that module from the package. As an example, a package may house a plurality of module and fluid delivery device assemblies 102, 104 (e.g., a first assembly, a second assembly, etc.). Each of the assemblies of the plurality of assemblies 102, 104 (e.g., the first assembly, the second assembly, etc.) may be configured to be automatically activated (e.g., turned on, powered up, etc.) in response to an opening of the package and/or a removal of that module from the package.

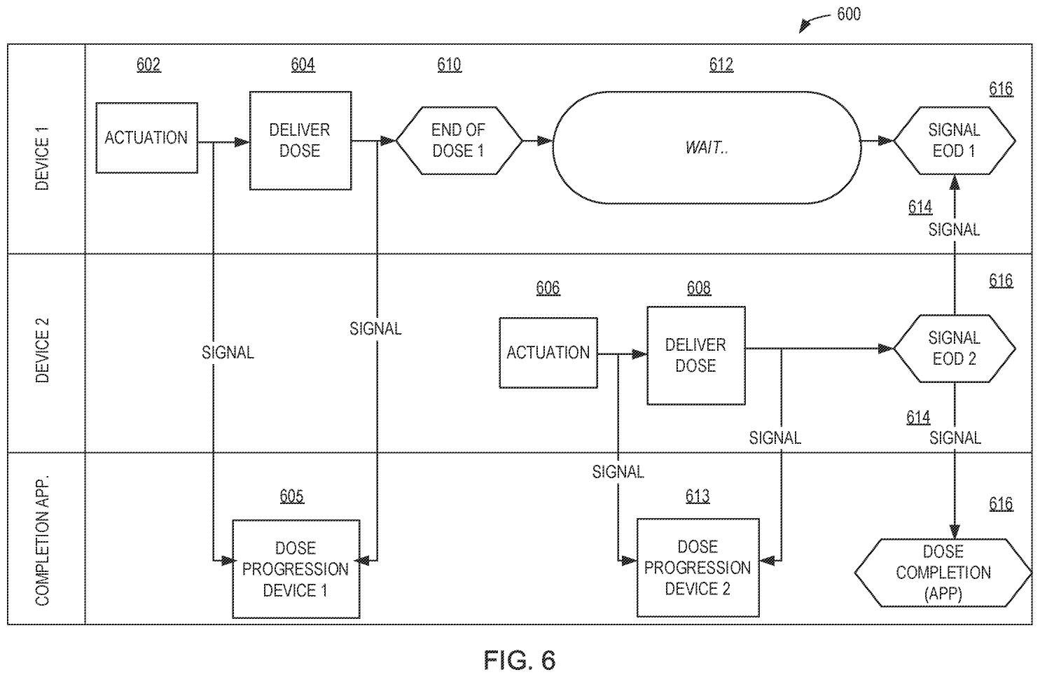

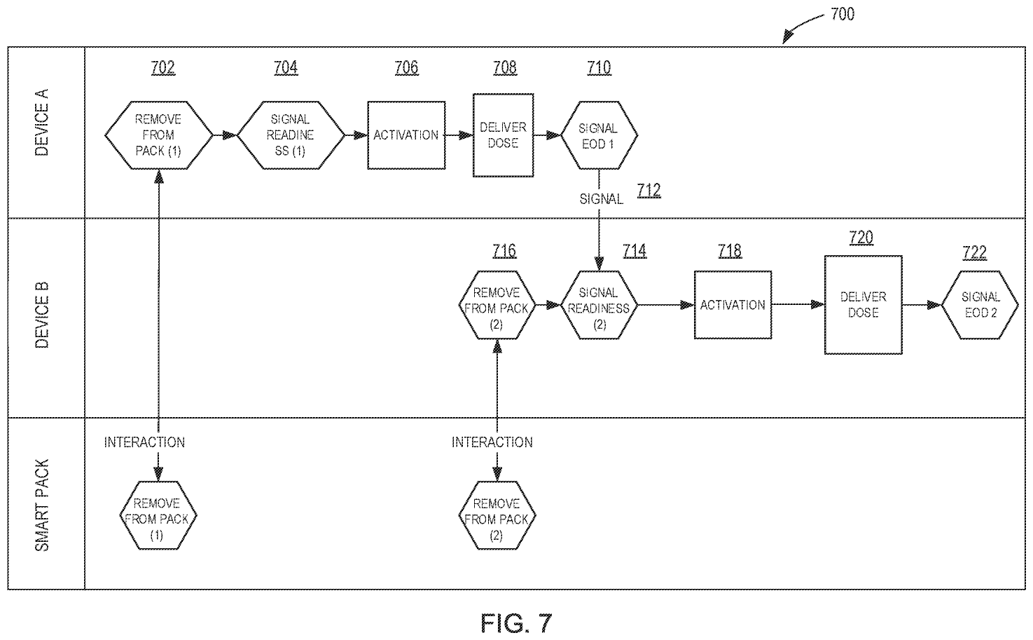

Further details regarding non-limiting embodiments or aspects of step 502 of process 500 are provided below with regard to FIGS. 6 and 7.

As shown in FIG. 5, at step 504, process 500 includes establishing a wireless communication network including the plurality of modules. For example, the plurality of modules 102 associated with the plurality of fluid delivery devices 104 (e.g., a plurality of assemblies 102, 104, etc.) may establish a wireless communication network including the plurality of modules 102. In such an example, the wireless communication network may include an ad-hoc mesh network. For example, the wireless communication device 214 in each module of the plurality of modules 102 may broadcast to, and receive signals from each of the other module(s) of the plurality of modules 102. As an example, in response to being activated, the plurality of modules 102 may self-organize into an ad-hoc mesh network, thereby establishing an electronic wireless signaling system.

For a parallel administration or configuration, for each module of the plurality of modules 102, in response to being activated, that module may: determine whether that module is a first module of the plurality of modules 102 to be activated; in response to determining that that module is the first module to be activated, establish that module as a master module (e.g., Device 1 in FIG. 6); and in response to determining that that module is not the first module to be activated, establish, with that module as a slave module (e.g., Device 2 in FIG. 6), the wireless communication network with the master module. As an example, for each module of the plurality of modules 102, that module may be activated (e.g., automatically activated, etc.) in response to an initiation of a fluid delivery procedure with the fluid delivery device associated with that module. In such an example, a module may be activated in response to a user pressing the injector activation button 356 of the fluid delivery device associated with that module. As an example, for each module of the plurality of modules 102, that module may be activated (e.g., automatically activated, etc.) in response to response to an opening of a package including that module and/or a removal of that module from the package.

For a parallel administration or configuration, in response to activating the plurality of modules 102 associated with the plurality of fluid delivery devices 104 (e.g., a plurality of assemblies 102, 104, etc.), the plurality of modules 102 associated with the plurality of fluid delivery devices 102 may include a master module associated with a first fluid delivery device (e.g., Device 1 in FIG. 6) and at least one slave module associated with at least one second fluid delivery device (e.g., Device 2 in FIG. 6).

For a serial administration or configuration, the plurality of modules 102 associated with the plurality of fluid delivery devices 104 may include a first module associated with a first fluid delivery device (e.g., Device A in FIG. 7, a first assembly 102, 104, etc.) and a second module associated with a second fluid delivery device (e.g., Device B in FIG. 7, a second assembly 102, 104, etc.). The first module may be activated and, in response to the activation of the first module, the first module may automatically provide (e.g., via the user feedback device of the first module, etc.) feedback indicating that the first fluid delivery device is ready for use in a first fluid delivery procedure. The second module may be activated and, in response to the activation of the second module, the second module may automatically establish the wireless communication network with the first module (e.g., with the first module as a master module and the second module as a slave module, etc.).

Further details regarding non-limiting embodiments or aspects of step 504 of process 500 are provided below with regard to FIGS. 6 and 7.

As shown in FIG. 5, at step 506, process 500 includes sensing states of the plurality of fluid delivery devices. For example, for each module of the plurality of modules 102, that module may sense (e.g., with sensor 210, etc.) a state of a fluid delivery device of the plurality of fluid delivery devices 104 associated with that module (e.g., each assembly 102, 104 of a plurality of assemblies 102, 104 may sense its state, etc.). As an example, for each module of the plurality of modules 102, that module may sense as the state of the fluid delivery device associated with that module at least one of the following: an initiation of a fluid delivery procedure or injection, a progression of the fluid delivery procedure or injection, a completion of the fluid delivery procedure or injection, or any combination thereof.

For a serial administration or configuration, the first module may sense the state of the first fluid delivery device (e.g., Device A in FIG. 7, a first assembly 102, 104) includes a completion of the first fluid delivery procedure (e.g., an end of dose, etc.) and, in response to sensing that the state of the first fluid delivery device includes a completion of the first fluid delivery procedure, the first module may communicate to the second module a message indicating that the first fluid delivery procedure is complete. In response to receiving the message indicating that the first fluid delivery procedure is complete, the second module may automatically provide, via the user feedback device of the second module, feedback indicating that the second fluid delivery device (e.g., Device B in FIG. 7, a second assembly 102, 104) is ready for use in a second fluid delivery procedure.

Further details regarding non-limiting embodiments or aspects of step 506 of process 500 are provided below with regard to FIGS. 6 and 7.

As shown in FIG. 5, at step 508, process 500 includes providing feedback associated with the sensed states. For example, one or more modules of the plurality of modules 102 may provide feedback associated with one or more states of one or more fluid delivery devices of the plurality of fluid delivery devices 104 (e.g., one or more assemblies of the plurality of assemblies 102 104 may provide feedback associated with its one or more states, etc.). As an example, the plurality of modules 102 may coordinate, via the wireless communication network, the feedback provided via each module (e.g., the feedback provided via the user feedback device 212 of each module, etc.) based on the sensed state of each fluid delivery device of the plurality of fluid delivery devices 104. For example, each module of the plurality of modules 102 may display consistent feedback to a user of the entire system (e.g., each activated module, etc.) in accordance with an injection progression across the plurality of fluid delivery devices 104 in accordance with a desired medication delivery sequence.

The plurality of modules 102 may be configured to enable serial and/or parallel administration of a fluid or medication with the plurality of fluid delivery devices 104, as may be predetermined for a specific molecule provided in the system as a combination product. Each of parallel and serial administration options are enabled by non-limiting embodiments or aspects by the present disclosure, as each option may be desired based on pharmacokinetics and/or usability considerations related to the drug, a disease, a user, a use environment, and/or a planned indication(s).

For a parallel administration or configuration, the plurality of modules 102 may delay providing via each module (e.g., via the user feedback device 212 of each module, etc.) an indication that the fluid delivery procedure associated with that module (e.g., associated with that assembly 102, 104, etc.) is complete until the fluid delivery procedure associated with each module of the plurality of modules 102 (e.g., associated with each assembly 102, 104, etc.) is complete. For example, the master module may delay providing (e.g., via the user feedback devices of the master module and the at least one slave module, etc.) (i) feedback indicating that a first fluid delivery procedure associated with the first fluid delivery device is complete and (ii) feedback indicating that at least one second fluid delivery procedure associated with the at least one second fluid delivery device is complete until (i) a state of the first fluid delivery device sensed by the master module includes a completion of the first fluid delivery procedure and (ii) a state of the at least one second fluid delivery device sensed by the at least one slave module includes a completion of the at least one second fluid delivery. As an example, a first module among the plurality of modules 102 to be activated may be identified, the first or activated module may listen for additional modules among the plurality of modules 102 to be activated, display user feedback during the injection process, and/or control display of coordinated user feedback (e.g., end of dose, etc.) on each meshed module only when each fluid delivery device corresponding to each module in the mesh network has completed its fluid injection. In this way, a user may be inhibited or prevented from removing an incorrect fluid delivery device(s) prior to injection completion and/or misinterpreting end-of-dose feedback from one fluid delivery device among the plurality of fluid delivery devices 104 (e.g., due to mechanical variability in one or more of the devices, etc.).

In some non-limiting embodiment or aspects, in a parallel administration or configuration, in response to activating the plurality of modules 102 associated with the plurality of fluid delivery devices 104, the master module may control the user feedback devices 212 of the master module and the at least one slave module to provide a same feedback indicating that a fluid injection is in progress until (i) the state of the first fluid delivery device sensed by the master module includes the completion of the first fluid delivery procedure and (ii) the state of the at least one second fluid delivery device sensed by the at least one slave module includes a completion of the at least one second fluid delivery.

For a serial administration or configuration, the plurality of modules 102 associated with the plurality of fluid delivery devices 104 (e.g., a plurality of assemblies 102, 104, etc.) may include a first module associated with a first fluid delivery device (e.g., a first assembly, etc.) and a second module associated with a second fluid delivery device (e.g., a second assembly, etc.). The second module may provide no feedback (e.g., no feedback via the user feedback device of the second module, etc.) until the second module receives, from the first module via the wireless communication network, a message indicating that the fluid delivery procedure associated with the first module is complete. For example, a first module in the series (e.g., in the plurality of modules 102, etc.) may be activated, the first module may display user feedback during the injection process of its corresponding fluid delivery device, control display of coordinated user feedback (e.g., instructions to identify a next fluid delivery device and/or start a fluid delivery procedure with the next fluid delivery device, etc.) on an appropriate next meshed module 102 corresponding to the next fluid delivery device, and/or repeating the process until each of the plurality of fluid delivery devices have been administered sequentially.

In some non-limiting embodiments or aspects, for a serial administration of configuration, the first module may sense that the state of the first fluid delivery device (e.g., Device A in FIG. 7, a first assembly 102, 104, etc.) includes an initiation of the first fluid delivery procedure and/or a progress of the fluid deliver procedure and, in response to sensing that the state of the first fluid delivery device includes an initiation of the first fluid delivery procedure, the first module may automatically provide (e.g., via the user feedback device 212 of the first module, etc.), feedback indicating that the first fluid delivery procedure is initiated and/or a progression of the first fluid delivery procedure.

In some non-limiting embodiments or aspects, for a serial administration of configuration, the plurality of modules 102 associated with the plurality of fluid delivery devices 104 (e.g., a plurality of assemblies 102, 104, etc.) may further include at least one third module associated with at least one third fluid delivery device (e.g., at least one third assembly 102, 104, etc.). In such an example, in response to sensing that the state of the second fluid delivery device includes a completion of the second fluid delivery procedure, the second module may communicate to the at least one third module a message indicating that the second fluid delivery procedure is complete and, in response to receiving the message indicating that the second fluid delivery procedure is complete, the at least one third module may automatically provide (e.g., via the user feedback device 212 of the at least one third module, etc.), feedback indicating that the at least one third fluid delivery device is ready for use in at least one third fluid delivery procedure.

For either parallel or serial administration, feedback may be provided on one or more modules of the plurality of modules 102 via user feedback device 212 as a visual indicator, haptic feedback, audible tones, verbal prompts or instructions (e.g., playback of recorded audio that provides a verbal prompt or instruction when played back, etc.), or any combination thereof. For example, one or more modules of the plurality of modules 102 may provide a start of dose, an in process feedback, and/or an end of dose feedback to a user. Non-limiting embodiments or aspects of the present disclosure may accommodate inherent mechanical variability (e.g., injection time tolerance, etc.) that may be present in fluid delivery devices. The feedback provided may reduce or prevent user confusion that may take place if observing feedback provided by a single fluid delivery device among the plurality of fluid delivery devices 104 during use.

In some non-limiting embodiments or aspects, feedback provided on one or more modules of the plurality of modules 102 may be used to not only improve intuitiveness during use, but also to indicate elements of a predetermined medication sequence to a user prior to use. Accordingly, non-limiting embodiments or aspects of the present disclosure may provide enhanced feedback methods for communicating an injection site, an anatomic side (e.g., left/right), and/or a device order (e.g., 1, 2 . . . n), and such feedback may be coordinated between the plurality of modules 102 via the wireless communication network.

Further details regarding non-limiting embodiments or aspects of step 508 of process 500 are provided below with regard to FIGS. 6 and 7.

FIG. 6 is a signal flow diagram of an implementation 600 of non-limiting embodiments or aspects of a process for fluid delivery using multiple fluid delivery devices in a parallel configuration. For example, a user, such as a patient, may be provided with a package including the plurality of fluid delivery device 104 having the plurality of modules 102 associated therewith (e.g., a plurality of assemblies 102, 104, etc.), for example, two OBI devices each with a corresponding module as referenced in FIG. 6, referred to herein as Device 1 (e.g., a first assembly 102, 104, etc.) and Device 2 (e.g., a second assembly 102, 104, etc.). The packaging may suggest that each device or assembly is to be used per dosing interval. It is noted that the ordinal labeling of the devices with respect to FIG. 6 is a term of convenience and need not be established a priori during manufacturing unless desired. For example, a “first” device or assembly need not be the first device or assembly in the package; it may simply be denoted Device 1 by virtue of being first activated. Device 2, 3, . . . , n may be similarly denoted as such by order of activation by a user, which may enable flexibility that does not appear in existing fluid delivery devices and/or allow for a wide variety of medication scenarios to be administered. As an example, in FIG. 6, Device 1 denotes the first OBI device or assembly activated by the user, and Device 2 denotes the second OBI device or assembly activated by the user. In such an example, coordination of feedback may ensure that each of the two OBI devices or assemblies referenced with respect to FIG. 6 display end of dose feedback only after each of the two OBI devices or assemblies complete their respective fluid delivery procedures or injections.

Before activation of the two OBI devices or assemblies referenced with respect to FIG. 6, the two devices or assemblies may be unpackaged from the outer carton and arranged for use. Each injection site may be prepared with isopropyl alcohol according to the instructions for use (IFU) of the device. Device 1's needle shield may be removed, removing the adhesive liner, and Device 1 may be removably adhered to the skin of the patient over the desired injection site, according to the IFU. These use steps may be repeated a second time for Device 2.

As shown in FIG. 6, at reference number 602, Device 1 may be activated, for example, by a user pressing the injector activation button 356 of Device 1, while Device 2 (although removably adhered to the patient) may not yet be activated. As further shown in FIG. 6, activation of Device 1 begins, at reference number 604, the fluid delivery procedure or injection process of Device 1, delivering the fluid or medication to the patient. In response to the activation of Device 1, Device 1 may determine that it is the first device to be activated and establishes itself as the “master device”. After establishing itself as the master device, Device 1 waits (e.g., polls, broadcasts, etc.) for Device 2 to be activated while Device 1's fluid delivery procedure or injection proceeds.

As further shown in FIG. 6, at reference number 605, Device 1 may provide feedback to a user of the device or assembly (e.g., via its user feedback device 212, via a companion application on a remote computing device, etc.) indicating the fluid delivery procedure or injection of Device 1 is in process and/or a progress thereof. In some non-limiting embodiments or aspects, the plurality of modules 102 (e.g., Device 1, Device 2, etc.) may be configured for wireless external communication, such as using a Bluetooth, Zigbee, or Wi-Fi or cellular communication protocol, with an application on a remote computing device, such as a tablet, a mobile telephone, and/or a server-based application. The application on the remote device may be configured to display real-time data regarding the state and/or performance of one or more fluid delivery devices of the plurality of fluid delivery devices 104 (e.g., Device 1, Device 2, etc.).

The remote computing device may be configured to provide contextual instructions to a patient and/or user during use of the plurality of fluid delivery devices 104 (e.g., Device 1, Device 2, etc.). For example, the remote computing device may provide instructions to the patient and/or user on how to set up and initiate a dosing procedure using plurality of modules 102/plurality of fluid delivery devices 104 (e.g., Device 1, Device 2, etc.). In some examples, the remote computing device may indicate to the patient that a dosing procedure is ongoing and provide status indication of various stages of the dosing procedure. In further examples, the remote computing device may provide instructions to the patient on a procedure to be followed in an extraordinary event, such as in an instance when the dosing procedure may stall. The plurality of modules 102/plurality of fluid delivery devices 104 (e.g., Device 1, Device 2, etc.) may be configured to send information, using the remote computing device, to a third party, such as the patient's medical provider or medical insurance company about time, date, and volume of the therapeutic agent delivered to the patient. The plurality of modules 102/plurality of fluid delivery devices 104 (e.g., Device 1, Device 2, etc.) may contact such third party in case of an extraordinary event, such as by sending a text alert or dialing a telephone number of the third party.

Data from the plurality of modules 102/plurality of fluid delivery devices 104 (e.g., Device 1, Device 2, etc.) may be transmitted to the remote computing device in real time and/or the data may be stored in a remote database for post-delivery use. In some examples, the remote computing device may be used to run a safety protocol prior to when a dosing procedure is initiated. For example, the remote computing device can check for drug recalls, verify that the correct therapeutic agent is used, and/or verify the time and volume of the last dosing procedure. The plurality of modules 102/plurality of fluid delivery devices 104 (e.g., Device 1, Device 2, etc.) may be blocked from initiating a new dosing procedure depending on whether the safety protocol run on the remote computing device detects any abnormalities. In some examples, the application on the remote computing device may be configured to display any data associated with plurality of modules 102/plurality of fluid delivery devices 104 (e.g., Device 1, Device 2, etc.). In some examples, plurality of modules 102/plurality of fluid delivery devices 104 (e.g., Device 1, Device 2, etc.) may have a BLE/MCU radio for wireless external communication with the remote computing device.

As further shown in FIG. 6, at reference number 606, at some point, a user activates Device 2 (e.g., by a user pressing the injector activation button 356 of Device 2, etc.). As further shown in FIG. 6, activation of Device 2 may begin, at reference number 608, the fluid delivery procedure or injection process of Device 2, delivering the fluid or medication to the patient. Device 2 may establish a communication connection to Device 1, which has been listening since it was activated, such that Device 1 and Device 2 are in communicating state with each other. For example, each of Device 1 and Device 2 may communicate messages associated with a start of dose, an injection process feedback, and/or an end of dose to each other.

As Device 2 may be activated sometime after Device 1, it is likely Device 1 finishes its fluid delivery procedure or injection before Device 2 finishes its fluid delivery procedure or injection, assuming each device is configured with equal or similar medication volumes and internal fluidic paths (e.g., a fluid delivery rate may be the same for each device, subject to mechanical tolerance on the devices and inherent variability). However, as an add-on module 104 may shield a mechanically driven end of dose indicator for Device 1 and 2, at reference number 610, end of dose for Device 1 may not displayed to the user, even though Device 1 has completed its fluid delivery procedure. As Device 2's injection is not complete, injection with Device 2 proceeds, and neither device displays end of dose, with Device 1 waiting, at reference number 612, for an end of dose message from Device 2. As further shown in FIG. 6, at reference number 613, Device 2 may provide visual feedback to a user of the device or assembly (e.g., via its user feedback device 212, via a companion application on a remote computing device, etc.) indicating the injection is in process. Additionally, or alternatively, each of Device 1 and 2 may provide feedback to a user (e.g., via each user feedback device 212 of each device, etc.) indicating the injection is in process with both devices, thereby further error-proofing interpretation of the feedback and reducing or preventing premature removal of one of the devices.

At some point, Device 2 completes the injection. As further shown in FIG. 6, at reference number 614, Device 2 may communicate to Device 1 (and optionally the companion application) a message indicating that its dose is complete. As further shown in FIG. 6, at reference number 616, as the injection of Device 1 is also completed, each of Device 1 and Device 2 (and optionally the companion application) may signal end of dose simultaneously, for example, with a different colored LED, flashing LED, or solid LED. Note that Device 1 and 2 did not actually stop or end their injections at the same time; the end of dose feedback for individual devices may be suppressed by the system and only presented once both devices or assemblies have completed the injection. This is extensible to scenarios with three or more devices. Further, such a feedback scheme may be independent of fill volume in each fluid delivery device or assembly, which need not be identical in each device or assembly. For example, the plurality of fluid delivery devices 104 may not be the same size (e.g., a 5 mL and 10 mL device may be used, two 5 mL devices may be used, two 10 mL devices may be used, etc.).

FIG. 7 is a signal flow diagram of an implementation 700 non-limiting embodiments or aspects of a process for fluid delivery using multiple fluid delivery devices in a serial configuration. For example, a user, such as a patient, may be provided with a package including the plurality of fluid delivery device 104 having the plurality of modules 102 associated therewith (e.g., a plurality of assemblies 102, 104, etc.), for example, two OBI devices each with a corresponding module as referenced in FIG. 7, referred to herein as Device A (e.g., a first assembly 102, 104, etc.) and Device B (e.g., a second assembly 102, 104, etc.). Ordinal labeling of the devices A and B described with respect to FIG. 7 may be a function of serial administration, which may distinguish it from the ad hoc nature of the example parallel case discussed previously with respect to FIG. 6. With respect to FIG. 7, Device 1 denotes the first device or assembly to be activated by the user in sequence, and Device 2 denotes the second device or assembly to be activated by the user, also in sequence.

Devices A and B may be provided in a package that presents both devices or assemblies within view at the same time, thereby suggesting that both devices or assemblies are to be used per dosing interval and/or allowing visualization of the feedback described herein below. For example, the OBI devices or assemblies may be packaged in blister package, as with a thermoformed blister and Tyvek® lidstock.

The package may be opened (e.g., by removing the lidstock from the blister either covering Device A or each of Devices A and B, etc.). In response to the opening of the package Device A and Device B may be activated (e.g., powered on, etc.). As shown FIG. 7, at reference number 702, Device A may be removed from the package and Device A may automatically provide feedback to the user (e.g., playback of a recorded verbal message indicating a number of devices to be used in a dosing regimen, a predetermined time until Device A is ready for dosing, etc.) and, at reference number 704, Device A may automatically provide feedback to the user indicating that Device A is ready to use (e.g., a recorded verbal message instructing the user to place Device A at a particular location on the user's body and to actuate the device when ready, etc.). In response to the activation of Device A, Device A may determine that it is the first device to be (correctly) activated for the serial administration, establish itself as the “master device”, and put Device B in a “listen only” mode.

An injection site A for Device A may be prepared with isopropyl alcohol per the IFU of the device. Device A's needle shield may be removed, removing the adhesive liner, and Device A may be removably adhered to the skin of the patient over the desired injection site A, also according to the IFU of the device. As further shown in FIG. 7, at reference number 706, the fluid delivery procedure or injection of Device A may be actuated (e.g., by a user pressing the injector activation button 356 of Device A, etc.), while Device B may remain in the package and/or remain silent (e.g., provide no audible, tactile, or visual feedback) in a “listen only” mode. As further shown in FIG. 7, at reference number 708, the fluid or medication of Device A may be delivered to the patient in the fluid delivery procedure or injection of Device A as configured.

As further shown in FIG. 7, at reference number 710, in response to a completion of the injection with Device A, an end of dose indicator may be provided on Device A (and optionally the companion application) (e.g., using visual, audible, and/or tactile feedback, such as play back of a recorded verbal message indicating that Device A has completed dosing and the user should remove and safely discard of Device A, and/or the like, etc.) and, at reference number 712, Device A may signal (e.g., via the wireless communication network, etc.) to Device B that the injection is complete, causing Device B, at reference number 714, Device B to provide an indication to the user that Device B is ready for use (e.g., using visual, audible, and/or tactile feedback, etc.).

As further shown in FIG. 7, at reference number 716, Device B may be removed from the package and arranged for use. An injection site B for Device B may be prepared with isopropyl alcohol per the IFU. Device B's needle shield may be removed, removing the adhesive liner, and Device B may be removably adhered to the skin of the patient over the desired injection site, also according to the existing IFU. As further shown in FIG. 7, at reference number 718, the fluid delivery procedure or injection of Device B may be actuated (e.g., by a user pressing the injector activation button 356 of Device B, etc.). As further shown in FIG. 7, at reference number 720, the fluid or medication of Device AB may be delivered to the patient in the fluid delivery procedure or injection of Device B as configured.

As further shown in FIG. 7, at reference number 722, in response to a completion of the injection with Device B, an end of dose indicator may be provided on Device B (and optionally the companion application) (e.g., using visual, audible, or tactile feedback, etc.). If additional devices are present, the process may repeat. If Device B is the last device in the serial sequence, Device B may be removed from the skin and the serial sequence may conclude.

Non-limiting embodiments or aspects of the present disclosure recognize a need to maintain continuity of packaged devices or assemblies to ensure that each device or assembly is used in a single dosing interval, which may mitigate risks that users “split” dosing across multiple dosing intervals and may be particularly relevant for biologics with comparatively shorter half-lives, and/or for pharmacokinetic considerations that require multiple doses to be administered in a single dosing interval. Further, non-limiting embodiments or aspects of the present disclosure may provide enhanced packaging for communicating permitted injection sites, anatomic side, instructions on site rotation between injections, a permitted time between a start of each injection, and/or an order of application to a user. Such enhanced packaging may also be used to reduce storage space of multiple devices or assemblies in a refrigerator, present multiple devices or assemblies to patients in an intuitive manner, and/or activate devices or assemblies.

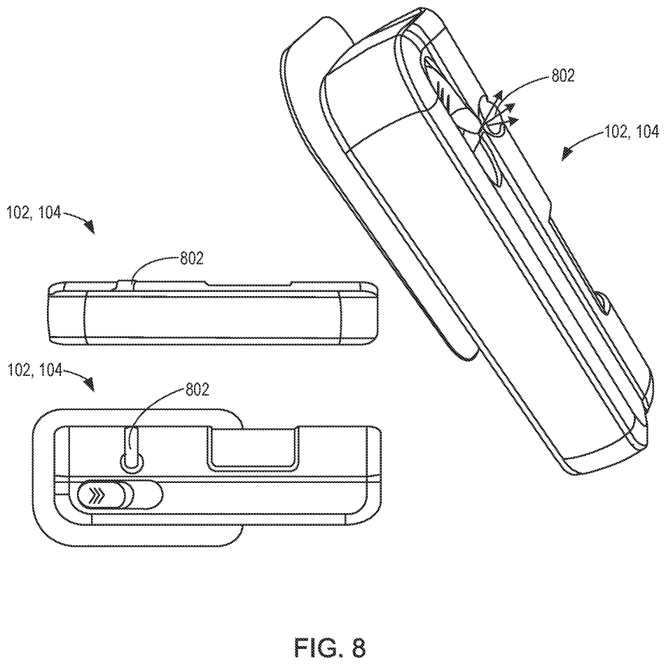

Referring now to FIG. 8, FIG. 8 is a perspective and side views of non-limiting embodiments or aspects of a module and fluid delivery device assembly. As shown in FIG. 8, a module and fluid delivery device assembly 102, 104 may include an electronic indicator 802. For example, a user feedback device 212 of the module and fluid delivery device assembly 102, 104 may include the electronic indicator 802, which may have an obround shape. As an example, the electronic indicator 802 may include light-piping illuminated via an LED to facilitate use in multiple orientations. In such an example, a variety of lensing approaches may be used to improve visibility and/or provide light to multiple locations on the module and fluid delivery device assembly 102, 104.

In some non-limiting embodiments or aspects, the module and fluid delivery device assembly 102, 104 has a change in contour and/or relief to allow use of a larger, protruding light pipe for electronic indicator 802 that protrudes from an outer surface of a housing of the module and fluid delivery device assembly 102, 104 and/or that is visible from each of a side and a top of the module and fluid delivery device assembly 102, 104 as illustrated in FIG. 8, which may be advantageous to extend a field of view for a user of the module and fluid delivery device assembly 102, 104, and/or to accommodate scenarios where the module and fluid delivery device assembly 102, 104 may be inadvertently placed upside down on the abdomen of the patient contrary to the instructions for use. In such an example, the light piping of the electronic indicator 802 may extend and/or be located in any position on the upper surface of the module and fluid delivery device assembly 102, 104 that does not interfere with internal components of the module and fluid delivery device assembly 102, 104. Additionally, a thickness of the housing of the module and fluid delivery device assembly 102, 104 may be slightly raised to avoid interference.

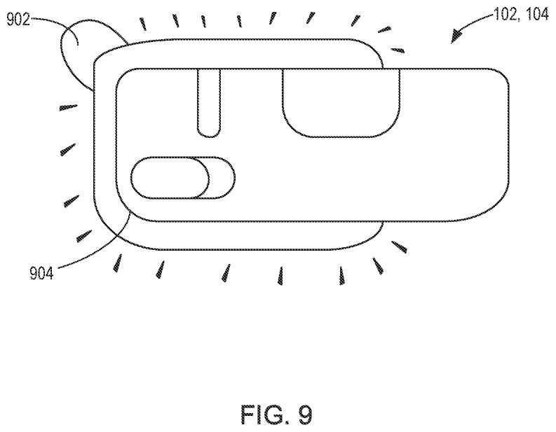

Additionally, or alternatively, as shown in FIG. 9, the light piping of electronic indicator 802 may extend down a sidewall of the module and fluid delivery device assembly 102, 104 from a top surface of the module and fluid delivery device assembly 102, 104 to an adhesive patch 902 of the module and fluid delivery device assembly 102, 104. The adhesive patch 902, which may be an opaque white material, may be used as a diffuser of light from the light pipe and/or may be provided with an additional clear flexible layer 904 for light piping, which may create a very large viewing surface for device-provided feedback that need not impact operation of the module and fluid delivery device assembly 102, 104 including the adhesive patch 902.

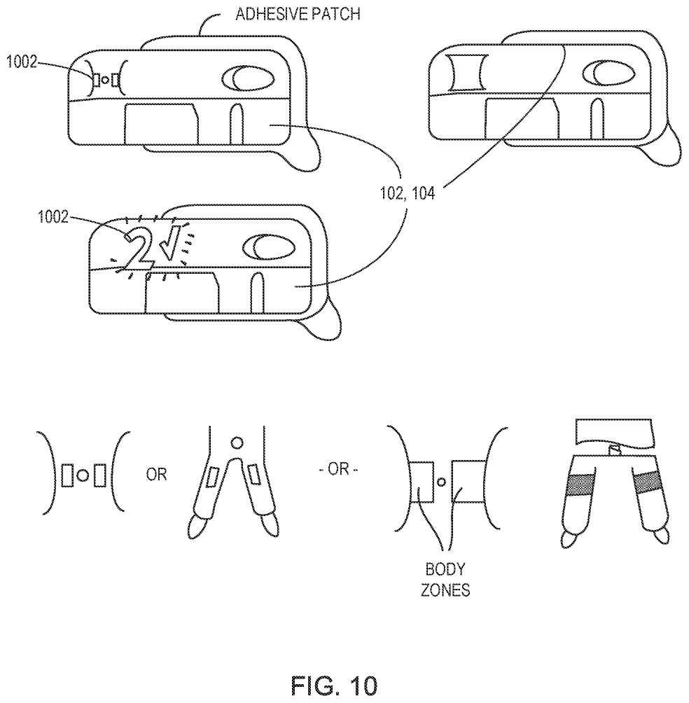

FIG. 10 is side views of non-limiting embodiments or aspects of a module and fluid delivery device assembly. As shown in FIG. 10, a module and fluid delivery device assembly 102, 104 may include a graphic 1002 formed from a thinned out section of the housing of the module and fluid delivery device assembly 102, 104 to reveal an image on the exterior of the assembly when backlit via an LED from within the housing. For example, the graphic 1002 may include silhouettes corresponding to body zones on the patient that are approved injection sites. As an example, the body zones may include the left and right abdomen and/or the left and right thigh. In such an example, the graphic 1002 including the body zones may be selectively illuminated at a beginning of preparation and/or extinguished once the injection proceeds to avoid confusion. Additionally, or alternatively, the graphic 1002 may include a silhouette that indicates an order of injection (e.g., 1, 2, n) and/or an injection side (e.g., left or right) when either the thigh or abdomen sites may be used for a particular module and fluid delivery device assembly 102, 104. In some non-limiting embodiments or aspects, the graphic 1002 may include a silhouette that may be illuminated in response to an end of dose or an injection being complete (e.g., a check or “X,” etc.), thereby providing a more intuitive indicator beyond a color-coded LED.

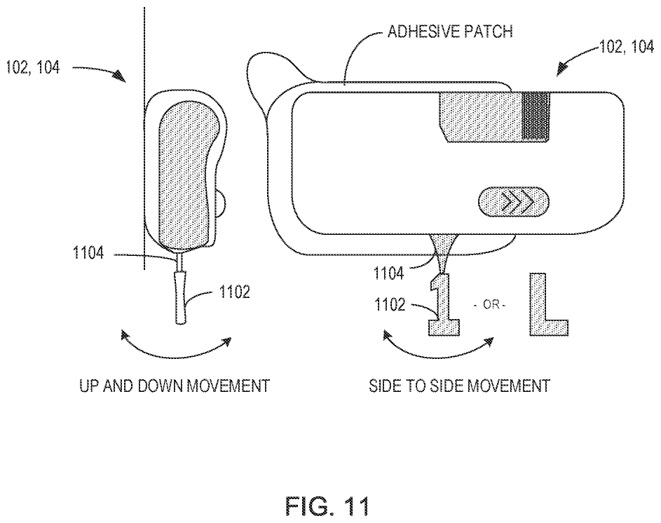

Although non-limiting embodiments or aspects of the present disclosure may be agnostic to device or assembly order, additional guidance that indicates a desired or proper order of use of a plurality of devices or assemblies may be provided. For example, as shown in FIG. 11, a module and fluid delivery device assembly 102, 104 may include a tag 1102, which may act as an ordinal indicator and/or a side-of-body indicator for a particular module and fluid delivery device assembly 102, 104. For example, the tag 1102 may extend from a housing of the module and fluid delivery device assembly 102, 104 (e.g., from a rubber grip thereof, etc.) and/or may include a flexible indicator that is shaped to indicate the ordinal position of that particular module and fluid delivery device assembly 102, 104 and/or a side of the body to which that particular module and fluid delivery device assembly 102, 104 is intended to be applied.

The tag 1102 may be molded with a connection section 1104 as a hinge (e.g., a thinner section between tag 1102 and assembly 102, 104, etc.) that allows movement (e.g., up and down and/or side to side relative to the assembly 102, 104), thereby providing user access to an adhesive strip of the assembly 102, 104 and/or attachment to the patient without interference. Although shown in FIG. 11 on an underside of the adhesive patch of assembly 102, 104, the tag 1102 may located and/or extend from any location on an exterior surface of the housing of the assembly 102, 104. The tag 1102 may include variety of indicia, such as a sequence number (e.g., “1”, “2”, etc.), an anatomic side or location (e.g., “L” for left, “R” for right, etc.), and/or the like. The tag 1102 may have a different color and/or a different material (e.g., a rigid plastic, etc.) from a flexible rubber grip of the assembly 102, 104, as through two-shot molding of the rubber components. The connection section or hinge 1104 may be tapered and/or provided with a variable cross section to provide more or less rigidity in a selected direction. For example, the connection section 1104 may be configured to flex out of the way of the adhesive patch in an up and/or down direction, while having more resistance to motion in side-to-side direction perpendicular to the up and/or down direction. In some non-limiting embodiments or aspects, the tag 1102 and/or the connection section or hinge 1104 may include an internal light pipe that may be illuminated by an LED within assembly 102, 104. For example, the tag 1102 may be used as a visual indicator (e.g., an illuminated “1” or “L”) to a user of the assembly 102, 104.

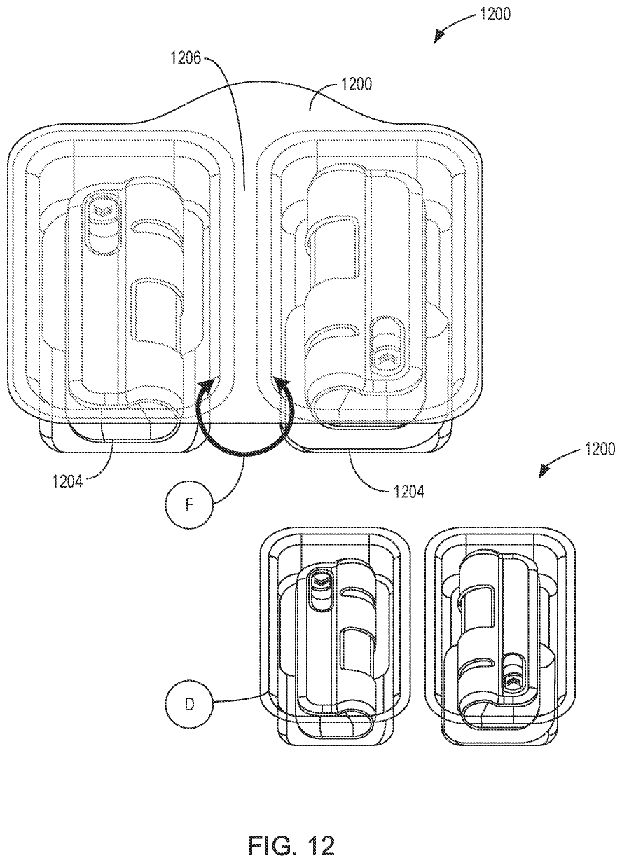

FIG. 12 is perspective view of non-limiting embodiments or aspects of a package for multiple module and fluid delivery device assemblies. As shown in an upper portion of FIG. 12, a package 1200 for multiple module and fluid delivery device assemblies 102, 104 may include a top sealing surface 1202 on a plurality of blisters 1204. A module and fluid delivery device assembly 102, 104 of a plurality of module and fluid delivery device assemblies 102, 104 configured to be administered together in a same dosing interval may be received in each blister 1204. Such a configuration may maintain a linkage between the plurality of module and fluid delivery device assemblies 102, 104 configured to be administered together, for example, when, as shown in FIG. 12, two devices are used (e.g., a left device and a right device, etc.), and/or may avoid internal contamination of the assemblies 102, 104 in storage and/or from secondary packaging particulate, which may improve maintenance of a sterile fluid path.

The top sealing surface 1202 may provide a tether 1206, which may act as a hinge, between the blisters 1204 when attached the blisters 1204, which may retain the assemblies 102, 104 in the package, communicate “togetherness”, and/or reinforce a notion that each of the assemblies 102, 104 are to be used in the same dosing interval. During preparation of the assemblies 102, 104, the top sealing surface 1202 may be removed in a single pull, with the devices retained in each blister 1204, which may include a retention feature, such as a press fit with an assembly 102, 104, (e.g., bumps, bosses extending into an interior of the blister 1204, etc.) that requires a positive effort or force from the user to remove the assembly 102, 104 from the blister 1204.

A secondary outer package (e.g., a card box, etc.) is not shown, but may be provided for housing the package 1200. The tether 1206 including a hinge may be advantageous when used with a functional secondary card box, which may present the assemblies 102, 104 in an angled presentation (e.g., standing up, laying flat, etc.).

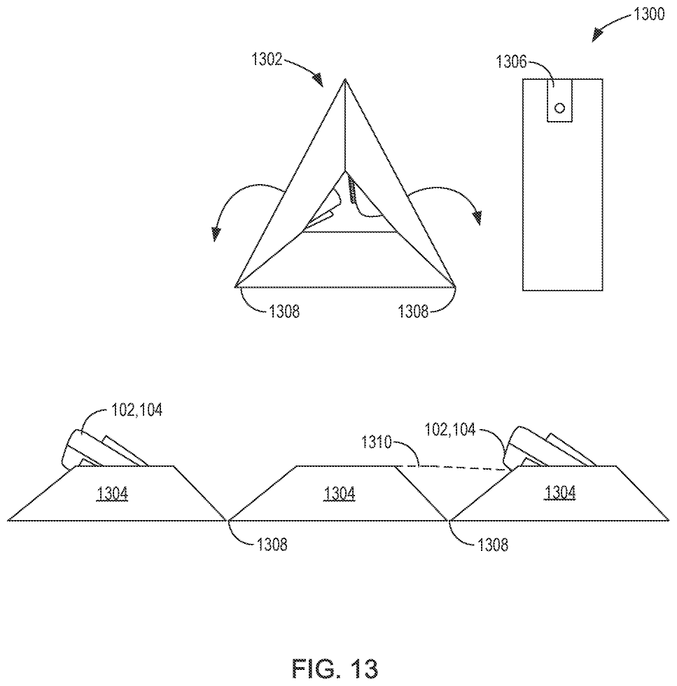

Referring now to FIG. 13, in some non-limiting embodiments or aspects, a package 1300 for multiple module and fluid delivery device assemblies 102, 104 may include an outer carton 1302 including a plurality of module and fluid delivery device assemblies 102, 104 that are separately packaged in a plurality of blisters for administration together in a same dosing interval. For example, as shown in FIG. 13, the outer carton 1302 may have a triangular shape formed by a series of segments or sides 1304 (e.g., a middle segment between two outer segments, etc.). For example, the outer carton 1302 may house an assembly 102, 104 at two of three equal segments or sides 1304 as shown in FIG. 13.

A closure 1306 (e.g., a button closure, a snap closure, a latch closure, etc.) may hold the two outer segments 1304 at opposite ends of the series of segments together. Unfastening the closure at H and unfolding triangular case about hinges 1308 may result in each of the three segments 1304 laying flat or horizontal as shown in the bottom portion of FIG. 13. The two assemblies 102, 104 may be retained within the outer two segments of the series of segments 1304 at an angle such that the button and needle shield are hidden, thereby inhibiting or preventing premature grasping and/or adhesive liner removal. An end of an assembly 102, 104 may protrude from the package 1300 when laid flat in an accessible and safe manner for a user to grab onto. The middle segment of the series of segments 1304 may include or house instructional materials and/or ancillary injection supplies, such as a product leaflet, alcohol swabs, and/or the like. A bridge 1310, which may be formed of a flexible material, may connect the segments (e.g., connect each of the outer two segments to the middle segment, etc.). The bridge 1310 may provide additional space for instructional content and/or a surface to place injection materials. When unfolded, the bridge 1310 may be tensioned to inhibit or prevent package 1300 from extending beyond the horizontal orientation shown at the bottom of FIG. 13. The series of segments may form truncated triangular prisms such that that there is a central cavity I through which the enclosed assemblies 102, 104 are visible when the package 1300 is in the closed position.

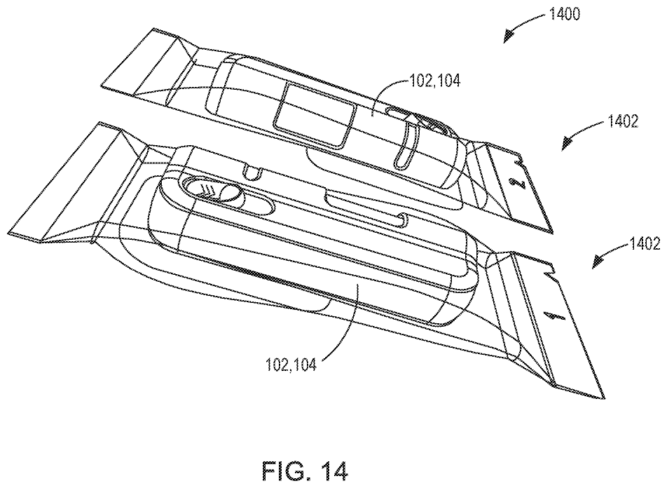

Referring now to FIG. 14, in some non-limiting embodiments or aspects, a package 1400 for multiple module and fluid delivery device assemblies 102, 104 may include multiple separate blisters 1402. For example, a plurality of module and fluid delivery device assemblies 102, 104 may be separately packaged in a plurality of separate blisters 1402 (e.g., a plurality of flexible blister packages with minimal conformal shape, which may be advantageously used to form a sterility barrier for other secondary packaging, etc.). The plurality of separate blisters 1402 may be packaged in outer carton (e.g., package 1300, package 1200, etc.), which may be configured to present the assemblies 102, 104 in a configuration that has selective accessibility of a portion of each assembly 102, 104 that is accessible and safe to grab onto, which may inhibit or prevent inadvertent use of a needle shield removal aid as a grab surface to remove an assembly 102, 104 from the carton. For example, use of a rigid needle shield pull ring as a handling point may inadvertently activate an assembly 102, 104 in an unanticipated or unwanted order.

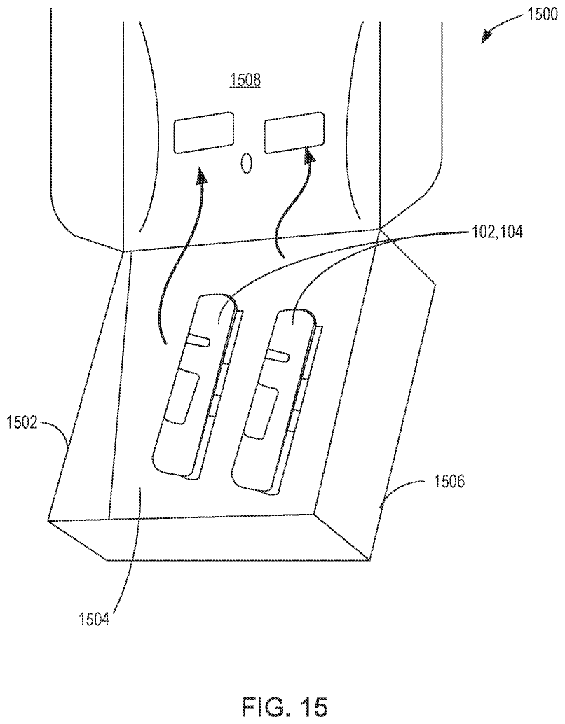

Referring now to FIG. 15, in some non-limiting embodiments or aspects, a package 1500 for multiple module and fluid delivery device assemblies 102, 104 may include a box 1502 including an interior floor 1504 that is angled relative to a bottom 1506 of the box 1502 to enable a user to be clearly be directed toward a front of assemblies 102, 104 contained within the box 1502 and/or to provide a clear grasping area for the assemblies 102, 104. Each assembly 102, 104 may be embedded edgewise into the inclined interior floor 1504 with a viewing window of each assembly 102, 104 facing up such that only one side of each assembly 102, 104 protrudes from the inclined interior floor 1504. Each assembly 102, 104 may be provided with a secondary package, such as a flexible blister, flowrap, and/or the like (e.g., blister 1402, etc.) to inhibit or prevent microbial contamination and/or preserve sterility of an internal fluid path within each assembly 102, 104. A portion of each assembly 102, 104 O that protrudes from the inclined floor 1504 and is visible and accessible within the box 1502 may be safe to grip and handle. To inhibit or prevent unintentional removal of a rigid need shield, a pull ring (not visible) of the rigid needle shield of each assembly 102, 104 may not be accessible for use as a handling point to remove each assembly 102, 104 from box 1502. Package 1500 may not reduce refrigerated storage space, but may be advantageous to present educational material to a patient using the device. Optional graphics and/or iconography 1508 may be placed on a lid of box 1502, which may show proper application of the assemblies 102, 104 to permitted injection sites. For example, as shown in FIG. 15, left and right orientations on an abdomen are shown; however, other concept variations may include thigh injection sites, and/or the like. Regardless, the lid of the box may be used as a “mirror image” of the patient and may be presented in a prominent position.

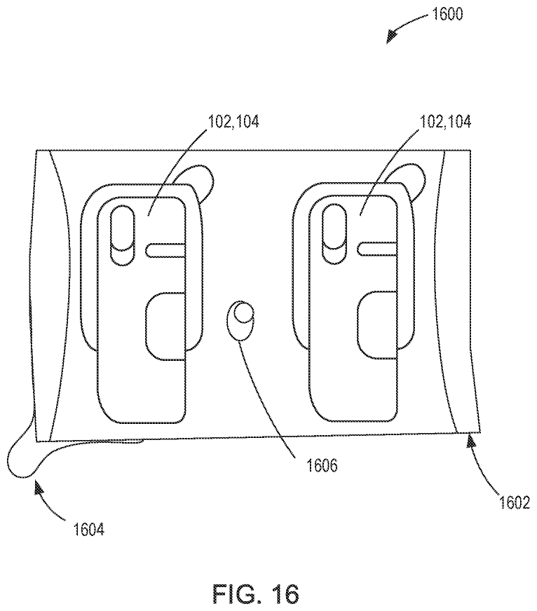

Referring now to FIG. 16, in some non-limiting embodiments or aspects, a package 1600 for multiple module and fluid delivery device assemblies 102, 104 may include a cover 1602 on one or more blisters 1604 housing a plurality of fluid delivery device assemblies 102, 104. The cover 1602 may include instructional indicia, such as anatomic placement instructions, and/or the like and/or other educational material. The assemblies 102, 104, which may be packaged in the same blister 1604 covered by the same cover 1602 or in multiple blisters 1604 covered by the same cover 1602, may help communicate that each assembly 102, 104 should be administered together (e.g., sequentially, in parallel, etc.) in a same administration or dosing interval. Cover 1602 may include an anatomical overlay and/or landmark 1606, such as the umbilicus shown in FIG. 16, depicting a permitted injection are on a body of the patient using the assemblies 102, 104. The assemblies 102, 104 may be oriented and positioned within the blister 1604 to create an illusion that the devices are positioned correctly to inject in the area depicted on the anatomical overlay 1606. For example, as shown in FIG. 16, left and right orientation on the abdomen may be provided; however, similar concept variations may include thigh injection sites, and/or the like. The anatomical overlay 1606 may be printed directly on the cover 1602 and/or may include an additional component printed on translucent paper and/or film, thereby allowing visualization of the anatomic sites while also allowing visualization of the assemblies 102, 104 within the blister 1604. The overlay may also comprise an underlay, for example, if the assemblies 102, 104 are packaged in transparent blisters and lid stock. Regardless, the cover 1602 may used as a “mirror image” of the patient and/or may be presented in a prominent position.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A system, comprising:
a plurality of modules associated with a plurality of fluid delivery devices, wherein each module of the plurality of modules includes: at least one processor, a wireless communication device configured to establish a wireless communication network with at least one other wireless communication device of at least one other module of the plurality of modules, at least one sensor configured to sense a state of a fluid delivery device associated with that module, and a user feedback device configured to provide feedback associated with one or more states of one or more fluid delivery devices of the plurality of fluid delivery devices;
wherein, for each module, in response to being activated, that module is configured to:
determine whether that module is a first module of the plurality of modules to be activated;
in response to determining that that module is the first module to be activated, establish that module as a master module; and
in response to determining that that module is not the first module to be activated, establish, with that module as a slave module, the wireless communication network with the master module.

2. (canceled)

3. The system of claim 21, wherein, for each module, that module is configured to be activated in response to an initiation of a fluid delivery procedure with the fluid delivery device associated with that module.

4. The system of claim 1, wherein, in response to activating the plurality of modules associated with the plurality of fluid delivery devices, the plurality of modules associated with the plurality of fluid delivery devices is configured to include a master module associated with a first fluid delivery device and at least one slave module associated with at least one second fluid delivery device, wherein the master module is configured to delay providing, via the user feedback devices of the master module and the at least one slave module, (i) feedback indicating that a first fluid delivery procedure associated with the first fluid delivery device is complete and (ii) feedback indicating that at least one second fluid delivery procedure associated with the at least one second fluid delivery device is complete until (i) a state of the first fluid delivery device sensed by the master module includes a completion of the first fluid delivery procedure and (ii) a state of the at least one second fluid delivery device sensed by the at least one slave module includes a completion of the at least one second fluid delivery.

5. The system of claim 4, wherein, in response to activating the plurality of modules associated with the plurality of fluid delivery devices, the master module is configured to control the user feedback devices of the master module and the at least one slave module to provide a same feedback indicating that a fluid injection is in progress until (i) the state of the first fluid delivery device sensed by the master module includes the completion of the first fluid delivery procedure and (ii) the state of the at least one second fluid delivery device sensed by the at least one slave module includes a completion of the at least one second fluid delivery.

6. The system of claim 1, wherein the plurality of modules associated with the plurality of fluid delivery devices includes a first module associated with a first fluid delivery device and a second module associated with a second fluid delivery device, wherein, in response to an activation of the first module, the first module is configured to automatically provide, via the user feedback device of the first module, feedback indicating that the first fluid delivery device is ready for use in a first fluid delivery procedure, and wherein, in response to an activation of the second module, the second module is configured to automatically establish the wireless communication network with the first module.

7. The system of claim 6, wherein, in response to sensing that the state of the first fluid delivery device includes a completion of the first fluid delivery procedure, the first module is configured to communicate to the second module a message indicating that the first fluid delivery procedure is complete, wherein, in response to receiving the message indicating that the first fluid delivery procedure is complete, the second module is configured to automatically provide, via the user feedback device of the second module, feedback indicating that the second fluid delivery device is ready for use in a second fluid delivery procedure.

8. The system of claim 7, wherein the second module is configured to provide no feedback via the user feedback device of the second module until the second module receives the message indicating that the first fluid delivery procedure is complete.

9. The system of claim 7, wherein, in response to sensing that the state of the first fluid delivery device includes an initiation of the first fluid delivery procedure, the first module is configured to automatically provide, via the user feedback device of the first module, feedback indicating that the first fluid delivery procedure is initiated and/or a progression of the first fluid delivery procedure.

10. The system of claim 7, wherein the plurality of modules associated with the plurality of fluid delivery devices further includes at least one third module associated with at least one third fluid delivery device, wherein, in response to sensing that the state of the second fluid delivery device includes a completion of the second fluid delivery procedure, the second module is configured to communicate to the at least one third module a message indicating that the second fluid delivery procedure is complete, wherein, in response to receiving the message indicating that the second fluid delivery procedure is complete, the at least one third module is configured to automatically provide, via the user feedback device of the at least one third module, feedback indicating that the at least one third fluid delivery device is ready for use in at least one third fluid delivery procedure.

11. The system of claim 6, further comprising:
a package housing the first module, the first fluid delivery device, the second module, and the second fluid delivery device,
wherein each of the first module and the second module is configured to be automatically activated in response to an opening of the package and/or a removal of that module from the package.

12. The system of claim 1, wherein the plurality of fluid delivery devices includes a plurality of on-body injectors.

13. The system of claim 1, wherein each module of the plurality of modules further includes a housing that houses the at least one processor, the wireless communication device, the at least one sensor, and the user feedback device, and wherein, for each module, the housing of that module is attached to a further housing of the fluid delivery device associated with that module.

14. The system of claim 13, wherein, for each module, the housing of that module covers a further user feedback device of the fluid delivery device associated with that module such that the further user feedback device is inhibited from providing feedback to a user.

15. The system of claim 1, wherein each module of the plurality of modules is integrated with the fluid delivery device associated with that module such that the at least one processor, the wireless communication device, the at least one sensor, and the user feedback device are included in a same housing of the fluid delivery device associated with that module.

16. The system of claim 1, wherein the wireless communication network includes an ad-hoc mesh network.

17. The system of claim 1, wherein each module of the plurality of modules is configured to sense as the state of the fluid delivery device associated with that module at least one of the following: an initiation of a fluid delivery procedure, a progression of the fluid delivery procedure, a completion of the fluid delivery procedure, or any combination thereof.

18. The system of claim 17, wherein the plurality of modules is configured to coordinate, via the wireless communication network, the feedback provided via the user feedback device of each module based on the sensed state of each fluid delivery device of the plurality of fluid delivery devices.

19. The system of 18, wherein the plurality of modules is configured to delay providing via the user feedback device of each module an indication that the fluid delivery procedure associated with that module is complete until the fluid delivery procedure associated with each module of the plurality of modules is complete.

20. The system of claim 18, wherein the plurality of modules associated with the plurality of fluid delivery devices includes a first module associated with a first fluid delivery device and a second module associated with a second fluid delivery device, and wherein the second module is configured to provide no feedback via the user feedback device of the second module until the second module receives, from the first module via the wireless communication network, a message indicating that the fluid delivery procedure associated with the first module is complete.

21. The system of claim 1, wherein the feedback includes a playback of a recorded verbal message associated with the one or more states of the one or more fluid delivery devices of the plurality of fluid delivery devices.

22. The system of claim 21, wherein one or more modules of the plurality of modules is configured to apply one or more feedback rules to the one or more states of the one or more fluid delivery devices of the plurality of fluid delivery devices to determine at least one module of the plurality of modules for providing the feedback associated with the one or more states of the one or more fluid delivery devices of the plurality of fluid delivery devices.

23. The system of claim 22, wherein the one or more feedback rules include at least one of the following feedback rules: a first feedback rule that a module of the plurality of modules associated with a fluid delivery device with a slower or longer delivery provides an end of dose feedback, a second feedback rule that a module of the plurality of modules associated with a first activated fluid delivery device of plurality of fluid delivery devices provides a start of dose feedback, or any combination thereof.

24. The system of claim 21, wherein the at least one sensor includes at least one of the following sensors: a start of dose sensor configured to sense a start of a dose by the fluid delivery device associated with that module, a dose progression sensor configured to sense a progression of the dose by the fluid delivery device associated with that module, an end of dose sensor configured to sense an end of the dose by the fluid delivery device associated with that module, a dose time and/or duration sensor configured to sense a time and/or a duration of the dose by the fluid delivery device associated with that module, a temperature sensor configured to sense a temperature of a fluid to be delivered by the fluid delivery device associated with that module, a needle shield removal sensor configured to sense a removal of a needle shield of the fluid delivery device associated with that module, an accelerometer configured to sense an orientation of the module and/or the fluid delivery device associated with that module, or any combination thereof.

25. The system of claim 21, wherein the at least one processor is configured to provide, as input to a machine learning model, the one or more states of the one or more fluid delivery devices of the plurality of fluid delivery devices, and receive, as output from the machine learning model, the feedback associated with the one or more states of the one or more fluid delivery devices of the plurality of fluid delivery devices.

26. The system of claim 21, wherein the playback of the recorded verbal message provides at least one of the following: an individual dose status of one or more fluid delivery devices of the plurality of fluid delivery devices; a combined dose status of the plurality of fluid delivery devices; a next user step; an instruction and/or a cautionary statement; a reminder for a next dose; or any combination thereof.

27.-60. (canceled)
